(19) United States
(12) Patent Application Publication
(10) Pub. No.: US 2026/0213015 A1
(43) Pub. Date: Jul. 23, 2026

(54) Multi-Modal Health Data Analysis and Response Generation System

(71) Applicant: Google LLC, Mountain View, CA (US)

(72) Inventors: Anastasiya Belyaeva, San Francisco, CA (US); Cory Yuen Fu McLean, Newton, MA (US); Tsz Ho Lee, Redmond, WA (US); Farhad Iraj Hormozdiari, Watertown, MA (US); Daniel Jonathan McDuff, Seattle, WA (US); Jian Cui, Fremont, CA (US); Justin Thomas Cosentino, Philadelphia, PA (US); Logan Douglas Schneider, Redwood City, CA (US); Nicholas A. Furlotte, San Francisco, CA (US); Shravya Ramesh Shetty, San Francisco, CA (US); Shruthi Prabhakara, Sunnyvale, CA (US); Shwetak Patel, Seattle, CA (US); Xin Liu, Seattle, WA (US); Yojan Patel, London (GB); Zhun Yang, San Francisco, CA (US)

(21) Appl. No.: 19/487,986

(22) PCT Filed: Mar. 19, 2024
(86) PCT No.: PCT/US2024/020607

§ 371 Date: Nov. 25, 2025

Related U.S. Application Data
(60) Provisional application No. 63/504,424, filed on May 25, 2023.

(51) Int. Cl. G16H 50/20; G16H 50/30

(52) U.S. Cl. CPC G16H 50/20; G16H 50/30

(57) ABSTRACT

Provided is a system for generating responses to health-related queries based on multi-modal health features of a patient. The responses generated by the system are conditioned on multi-modal health features associated with the patient, which can include one or more features from each of a plurality of modalities.

### RELATED APPLICATIONS

This application claims priority to and the benefit of U.S. Provisional Patent Application Number 63/504,424, filed May 25, 2023. U.S. Provisional Patent Application Number 63/504,424 is hereby incorporated by reference in its entirety.

### FIELD

The present disclosure relates to the intersection of machine learning and health informatics and, more specifically, to the processing and analysis of health-related data to generate personalized medical predictions and responses.

### BACKGROUND

Modern healthcare systems are increasingly reliant on the collection and analysis of health-related data to improve patient outcomes. However, there remains a significant challenge in effectively processing and analyzing this data to provide meaningful and personalized health insights. Traditional health informatics systems often struggle with integrating and interpreting data from various sources and modalities, such as combining textual clinical notes with quantitative sensor data. The inability to effectively synthesize this information can lead to suboptimal health predictions and recommendations, which may not fully reflect an individual's unique health profile.

For example, the vast majority of existing systems lack the capability to process time-series data, such as continuous heart rate monitoring, in a manner that can provide real-time or near-real-time feedback to users or healthcare providers. This limitation hinders the potential for early detection of health issues and timely intervention, which is beneficial in preventing adverse health events.

Additionally, there is a need for systems that can process health-related queries in natural language and generate responses that are both contextually relevant and personalized to the individual's health status. Existing systems often provide generic responses that may not take into account the full spectrum of an individual's health data, leading to a lack of personalization in health-related communication.

A technical problem to be solved is, therefore, to provide a system that can effectively integrate and analyze multi-modal health data, including time-series sensor data and textual information, to generate personalized health-related predictions and responses. The system should be capable of processing natural language queries to provide personalized, contextually relevant responses that reflect an individual's unique health profile.

### SUMMARY

Aspects and advantages of embodiments of the present disclosure will be set forth in part in the following description, or can be learned from the description, or can be learned through practice of the embodiments.

One general aspect includes a computer-implemented method for generating health-related predictions based on individual-level mobile health data. The computer-implemented method also includes obtaining, by a computing system may include one or more computing devices, mobile health data associated with an individual, where the mobile health data may include sensor data collected by a user computing device of the individual or derived from sensor data collected by the user computing device of the individual. The method also includes processing, by the computing system, the mobile health data with a sequence processing model to generate, as an output of the sequence processing model, a health-related prediction for the individual. The method also includes providing, by the computing system, the health-related prediction as an output. Other embodiments of this aspect include corresponding computer systems, apparatus, and computer programs recorded on one or more computer storage devices, each configured to perform the actions of the methods.

One general aspect includes a computing system may include one or more processors and one or more non-transitory computer-readable media that collectively store processor-executable instructions for performing operations. The operations include receiving a query relating to a particular individual. The operations include obtaining health features associated with the particular individual, where the health features may include one or more features from each of a plurality of modalities, and where, for at least one of the plurality of modalities, the health features are sensor data collected by a user computing device of the individual or are derived from sensor data collected by the user computing device of the individual. The operations include generating a respective representation of each of the multimodal health features as a respective set of one or more tokens in a natural language embedding space. The operations include generating an input sequence of tokens that may include one or more tokens in the natural language embedding space representing the query and the respective sets of one or more tokens in the natural language embedding space that represent each of the one or more features from each of the plurality of modalities. The operations include processing the input sequence of tokens using a sequence processing model to generate a model output. The operations include generating, from the model output, a health-related prediction that is responsive to the query. Other embodiments of this aspect include corresponding computer systems, apparatus, and computer programs recorded on one or more computer storage devices, each configured to perform the actions of the methods.

One general aspect includes a non-transitory computer-readable storage medium having stored thereon processor-executable instructions for performing operations. The operations include obtaining a set of health-related data from one or more data sources. The operations include prompting a large language model with the set of health-related data to generate an initial output may include one or more health-related predictions. The operations include receiving edits from one or more human experts to the initial output. The operations include submitting the edited initial output to a clinical lead for review and validation, thereby creating a validated response. The operations include combining the validated response with the set of health-related data to form a training tuple. The operations include training a sequence processing model on the training tuple, where the sequence processing model is configured to process and analyze multi-modal health data to generate personalized health-related predictions and responses when executed. Other embodiments of this aspect include corresponding computer systems, apparatus, and computer programs recorded on one or more computer storage devices, each configured to perform the actions of the methods. Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

FIG. 1 illustrates a schematic representation of a system for generating health-related predictions and responses based on individual-level mobile health data according to example implementations of aspects of the present disclosure.

FIG. 2 illustrates a schematic representation of a system for generating health-related predictions based on multi-modal health data according to example implementations of aspects of the present disclosure.

FIG. 3 illustrates a schematic representation of a system for generating health-related predictions based on multi-modal health data according to example implementations of aspects of the present disclosure.

FIG. 4 illustrates a schematic representation of a system for generating health-related predictions and responses according to example implementations of aspects of the present disclosure.

FIG. 5 illustrates a schematic representation of a system for generating health-related predictions and responses based on multi-modal health data according to example implementations of aspects of the present disclosure.

FIG. 6 illustrates a schematic representation of a system for generating training examples using human machine collaboration according to example implementations of aspects of the present disclosure.

FIG. 7 is a flow chart diagram illustrating an example method for training a machine-learned model according to example implementations of aspects of the present disclosure;

FIG. 8 is a block diagram of an example processing flow for using machine-learned model(s) to process input(s) to generate output(s) according to example implementations of aspects of the present disclosure;

FIG. 9 is a block diagram of an example sequence processing model according to example implementations of aspects of the present disclosure;

FIG. 10 is a block diagram of an example technique for populating an example input sequence for processing by a sequence processing model according to example implementations of aspects of the present disclosure;

FIG. 11 is a block diagram of an example model development platform according to example implementations of aspects of the present disclosure;

FIG. 12 is a block diagram of an example training workflow for training a machine-learned model according to example implementations of aspects of the present disclosure;

FIG. 13 is a block diagram of an inference system for operating one or more machine-learned model(s) to perform inference according to example implementations of aspects of the present disclosure;

FIG. 14 is a block diagram of an example networked computing system according to example implementations of aspects of the present disclosure;

FIG. 15 is a block diagram of an example computing device according to example implementations of aspects of the present disclosure; and

FIG. 16 is a block diagram of an example computing device according to example implementations of aspects of the present disclosure.

### DETAILED DESCRIPTION

Example aspects of the present disclosure are directed to computer-implemented systems and methods for generating responses to health-related queries based on multi-modal health features of a user (e.g., a medical patient). Some example systems can be implemented in various ways, such as a user-facing health and wellness chatbot or “personal health agent”, a clinician assistant software, and/or an early warning system in a medical treatment facility.

Some example systems can use a sequence processing model, a type of neural network, to process an input sequence of tokens that represent the query or other input and the multi-modal health features to produce an output sequence of tokens that represent a response to the query or other input. One example type of sequence processing model is a so-called large language model (LLM).

In some implementations, the multi-modal health features can include text features, image modalities, time series data modalities, tabular modalities, and/or other modalities of data. These features can be generated from a variety of sources, including demographic information, clinical information, medical images, laboratory test results, mobile health data collected or derived from sensors on the user's computing device, and/or other sources of data. Some example systems can represent each of these health feature as a set of one or more tokens in a natural language embedding space, even if the features are not represented as natural language.

One of the problems with prior techniques is the inability to provide personalized responses to health-related queries. The present disclosure addresses this issue by providing responses that are conditioned on multi-modal health features, allowing for responses that are specific to the current patient's health status. Another advantage is the system's ability to leverage the capabilities of sequence processing models for personalized health tasks, enabling it to ingest a diversity of data modalities relevant to an individual's health status.

In some implementations, the system can also include mobile health data sourced from a user's computing device, such as a smartphone or wearable device. This data can be processed to generate personalized health-related predictions. Furthermore, the system can handle a wide array of health-related data inputs, providing a comprehensive view of an individual's health and wellness status.

The sequence processing model can be trained on a variety of data types, enhancing its ability to predict health-related outcomes from mobile health data. As one example, the sequence processing model can be trained on high-quality case studies created through a collaborative process involving an initial generation of outputs by a large language model, subsequent refinement by human experts, validation by a clinical lead, and the formation of training tuples that combine this expert-validated advice with health data. This training ensures the model's outputs are accurate, personalized, and reflective of expert medical knowledge.

More particularly, the present disclosure describes a system for generating responses to health-related queries based on multi-modal health features of a patient. The responses generated by the system are conditioned on multi-modal health features associated with the patient, which can include one or more features from each of a plurality of modalities.

The multi-modal health features for a given patient can include features from multiple different modalities. These modalities can include text features, image modalities, time series data modalities, tabular modalities, and/or other modalities of features. For example, the text features can be generated from demographic information for the patient, clinical information for the patient, and so on. The image modality can represent a particular type of medical image of a portion of the patient's body. The time series modality can represent results across time of a particular type of laboratory test or diagnostic measurement for the patient. The tabular modality can represent various results of a particular type of laboratory test for the patient as a table.

In some implementations, the multi-modal health features can include mobile health data sourced from a user's computing device, such as a smartphone, wearable fitness tracker, or smartwatch. These devices are capable of collecting a wide range of health-related sensor data, which can then be processed to generate personalized health-related predictions, aligning with the system's capability to handle various health modalities. The mobile health data can include various types of raw sensor data and/or measures or other scores that are derived from raw sensor data. As one example, the mobile health data can include raw heart rate data collected over time. As another example, the mobile health data can include an aggregated score like a sleep score that has been precomputed from various raw data inputs using a predefined heuristic or algorithm. This flexibility demonstrates the system's ability to handle a wide array of health-related data inputs, providing a comprehensive view of an individual's health status.

The disclosed systems and methods can include and use a sequence processing model for analyzing the mobile health data, generating health-related predictions based on the collected data. For example, the model can predict sleep quality from sleep pattern data or create personalized workout recommendations from heart rate and step count data, demonstrating the adaptability of the system to different health data inputs.

In some implementations, the multi-modal health features can include or be represented using textual data. For example, tabular data can be reformatted into a sequence of textual tokens. This textual data enhances the accuracy of the predictions generated by the sequence processing model, illustrating the system's use of text features as part of the multimodal health features, which can include lifestyle factors such as diet and exercise habits.

Additionally or alternatively, various multi-modal health features can be represented in the natural language embedding space using encoder model(s) that operate to transform various data modalities into embeddings expressed within the natural language space. For example, these encoder model(s) can be appended to the sequence processing model and can operate to transform various modalities of data into a shared embedding space. For example, by setting a fixed number of vectors to represent each modality in the latent space, and training the modality-specific encoders while keeping the model weights frozen, both textual and non-textual data can contribute to the model's predictive accuracy.

As one example, some example methods can include transforming time-series data (e.g., sensor data) into embeddings using a time-series encoder model. This enables the system to represent all multi-modal input features in a natural language embedding space.

This transformation allows raw sensor data to be converted into a format that the sequence processing model can easily process, facilitating the integration of time-series data into the multi-modal health features.

Thus, to generate the response, the system can represent each feature of each modality as a respective set of one or more tokens in a natural language embedding space. This means that even if features of the modality are not represented as natural language, they can still be represented as tokens in the natural language embedding space. The system then generates an input sequence of tokens that includes one or more tokens representing the query and the respective sets of one or more tokens that represent each of the one or more features from each of the plurality of modalities.

The system processes the input sequence of tokens using a sequence processing model to generate a sequence processing model output. This sequence processing model is a type of neural network. The response can be a natural language sequence that is generated by the sequence processing model. Alternatively, the response can be a score that is computed based on output of the sequence processing model or a likelihood derived from the score. This score can indicate the likelihood that the patient has a particular medical condition specified in the query, the likelihood that the patient will suffer a particular adverse health event specified in the query within a threshold amount of time, the risk level of the patient developing the particular medical condition specified in the query, a predicted user-submitted response that would be received from the patient if the patient were provided with the query, health coaching information, and/or other forms of health-related predictions.

Thus, as examples, the health-related predictions generated can include user-submitted reports and health coaching messages, which are specific applications of the personalized responses to health-related queries generated by the system. These predictions are tailored based on individual data, such as sleep pattern data or a combination of heart rate data and step count data, showcasing the system's personalized approach.

Once the response is generated, the system can provide it for presentation to the user (e.g., patient) or to a clinician treating the patient. This means that the system can be used to provide personalized responses to health-related queries based on the individual health status of the user or patient. By conditioning the responses on the multi-modal user features, the system can provide responses that are specific to the current patient rather than generalized responses that may not be relevant to the current patient based on the patient's current health status.

The system can be implemented in a variety of ways. For example, it can be part of a user-facing health and wellness chatbot where the queries are received as input by the system from users. It can also be part of a clinician assistant software system where the queries are received as input by the system from clinicians or automatically generated by another component of the clinician software. Additionally, it can be part of an early warning software system employed by a medical treatment facility that generates suggestions for escalating patient care by predicting which patients'conditions are likely to clinically deteriorate.

The sequence processing model can be trained on a variety of data types, including professional examination data and patient-reported outcome data. This training enhances the model's ability to predict health-related outcomes from mobile health data, reflecting the system's adaptability to different training datasets.

As one example, in some implementations, the sequence processing model is enhanced through a sophisticated training regimen that leverages the collaborative efforts of human expertise and machine learning models. This training process is designed to refine the model's ability to generate accurate and reliable health coaching responses and other health-related outputs. The creation of the training data involves a multi-step process that begins with the acquisition of a comprehensive set of health data.

Once the health data is collected, it is utilized as a prompt for a large language model (LLM), which generates an initial output. This initial output may take the form of preliminary health coaching responses or other health-related advice. The role of the LLM at this stage is to provide a base output that captures the potential insights and recommendations that can be gleaned from the health data.

Following the generation of the initial outputs, one or more human experts, such as medical professionals or health data specialists, intervene to review and refine these outputs. Their expertise is beneficial in ensuring that the health advice is not only accurate but also tailored to the specific considerations of individual health scenarios. The experts may edit the outputs to enhance their clarity, relevance, and adherence to current medical guidelines and best practices.

After the initial outputs have been revised by the human experts, a clinical lead—a highly experienced medical practitioner—conducts a thorough review. The clinical lead's validation is an important step, confirming that the edited outputs meet the stringent standards required for medical accuracy and efficacy.

Once the outputs have been edited by the human experts and validated by the clinical lead, they are paired with the corresponding health data to form a training tuple. This training tuple represents a complete case study, encompassing both the raw health data and the expert-validated responses.

Finally, the sequence processing model is trained on these training tuples. The inclusion of edited and validated outputs in combination with the health data ensures that the model learns from high-quality, expert-curated information. This training approach not only improves the model's predictive accuracy but also its ability to provide personalized and contextually appropriate health coaching responses. By incorporating the insights of human experts into the training process, the sequence processing model becomes more attuned to the nuances of real-world health data and more capable of delivering outputs that can be trusted and acted upon by end-users.

The present disclosure provides several advantages. For example, it leverages the capabilities of sequence processing models (e.g., LLMs) to solve tasks across a wide range of fields. Example systems described in the present disclosure enable LLMs to ingest a diversity of data modalities that are relevant to an individual's health status, thereby making it possible to use LLMs for personalized health tasks. Furthermore, the system is capable of representing multiple modalities in the same natural language embedding space used by the LLM, which allows the LLM to effectively adapt for use in personalized health settings without needing further training.

The systems and methods of the present disclosure provide a number of technical effects and benefits. First, in some implementations, the systems and methods of the present disclosure can control a technical process within the healthcare domain by generating personalized health-related predictions and responses, thereby directly interacting with and affecting the operation of medical devices or healthcare systems. By generating personalized health-related predictions and responses based on multi-modal patient data, the systems and methods of the present disclosure are capable of interfacing with and providing directives to medical devices and systems. This control can extend to the adjustment of device settings, the triggering of alerts, and the initiation of diagnostic or therapeutic procedures, all of which are technical actions within the medical technology field.

Another technical effect results from providing a system and multi-modal model architecture and training approaches that effectively integrate and analyze multi-modal health data, including time-series sensor data and textual information, to generate personalized health-related predictions and responses. The system is capable of processing natural language queries to provide personalized, contextually relevant responses that reflect an individual's unique health profile.

Various example implementations are described herein with respect to the accompanying Figures.

### Example Multi-Modal Models and Training Processes

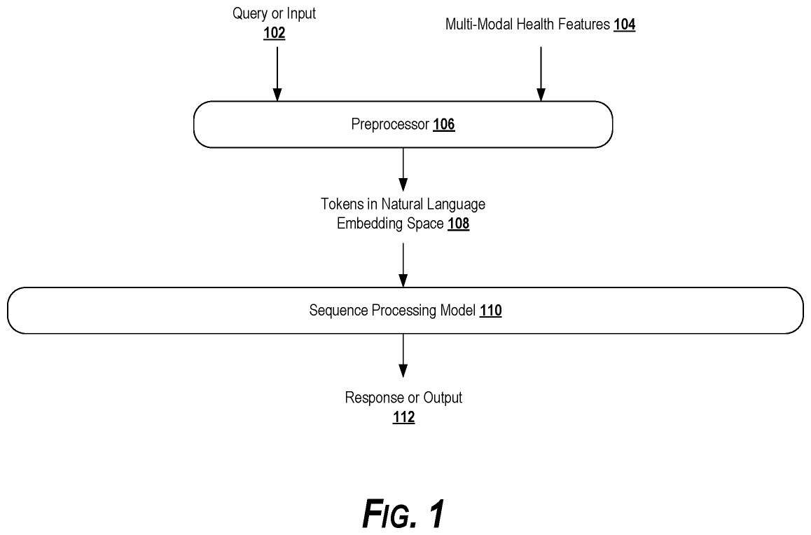

FIG. 1 illustrates a schematic representation of a system for generating health-related predictions and responses based on individual-level mobile health data according to the present disclosure. The system depicted in FIG. 1 can be implemented through a computing system comprising one or more computing devices that are configured to perform the operations described herein.

The system begins with a query or input 102, which can be a health-related query submitted by a user or a clinician. The query or input 102 can consist of natural language text, a structured form, or even verbal input that is subsequently converted to text. This query or input 102 can be specific to the individual's health status or a general inquiry related to health and wellness.

Adjacent to the query or input 102 are the multi-modal health features 104.

These features can be derived from various sources, including but not limited to, sensor data collected by a user computing device such as a smartphone or wearable device. The multimodal health features 104 can include a wide array of data types, such as time-series sensor data, tabular data, textual data, and clinical health features. These features can provide a comprehensive overview of an individual's health status, ranging from sleep patterns and heart rate variability to workout summaries and dietary habits.

The preprocessor 106 serves as a beneficial intermediary that receives both the query or input 102 and the multi-modal health features 104. The preprocessor 106 can perform various tasks, such as data cleaning, normalization, and transformation, to prepare the input and health features for further processing. It can convert the multi-modal health features 104 into a format that is amenable to analysis by the sequence processing model 110. This may include the transformation of raw sensor data into embeddings using a time-series encoder model or the conversion of tabular health data into textual tokens.

Tokens in the natural language embedding space 108 represent the transformation of the processed query or input 102 and multi-modal health features 104 into a format that is compatible with the sequence processing model 110. These tokens can be generated by the preprocessor 106 and are designed to encapsulate the semantic and contextual information contained within the query and health features. By representing these elements as tokens in a natural language embedding space, the system ensures that non-textual data such as sensor readings can be interpreted in conjunction with textual data within the same analytical framework.

The sequence processing model 110, which is a type of neural network, receives the tokens in the natural language embedding space 108 and processes them to generate a health-related prediction or response. The sequence processing model 110 can be a large language model (LLM) that has been trained on a diverse dataset, including professional examination data, patient-reported outcome data, and case studies validated by human experts. The model 110 can analyze the input sequence of tokens to infer patterns, correlations, and insights that inform the generation of the health-related prediction or response.

Finally, the response or output 112 is the output of the system's processing. This response or output 112 can take various forms, such as a natural language answer to the query, a health coaching message, a risk assessment, or a predictive user-submitted report. The response or output 112 is tailored to the individual's specific health data and the context of the query, ensuring that it is personalized and relevant. The response or output 112 can then be provided for presentation to the user or to a clinician treating the patient, thereby completing the system's cycle of generating health-related predictions and responses.

Additionally or alternatively, the response or output 112 can be used to control one or more healthcare machines or processes in a healthcare setting.

The system depicted in Figure I can be employed in various implementations, such as a user-facing health and wellness chatbot, a clinician assistant software, or an early warning system in a medical treatment facility. The flexibility of the system allows for a wide range of applications in the healthcare domain, leveraging the capabilities of sequence processing models to provide personalized and contextually relevant health-related predictions and responses based on multi-modal health data.

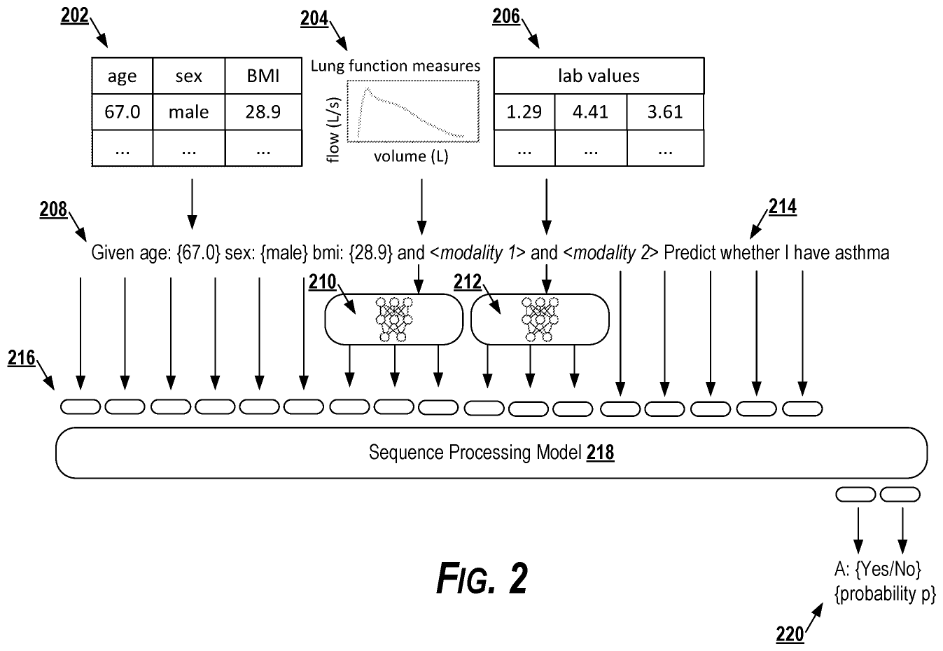

FIG. 2 illustrates a schematic representation of a system for generating health-related predictions based on multi-modal health data according to the present disclosure. This system can be implemented through a computing system comprising one or more computing devices configured to perform operations as described herein.

The system begins with tabular health features 202, which can include, for example, demographic information. These features can be input into the system in a structured format, such as a table or a database record. The tabular health features 202 provide foundational data that can be beneficial in determining the context of subsequent health-related analyses and predictions.

Adjacent to the tabular health features 202 are time-series health features 204, which can represent dynamic health data collected over time. This may include, for example, lung function measures depicted as flow-volume curves. The time-series health features 204 can be derived from a variety of sources, such as medical devices or sensors, and can provide insight into the temporal aspects of an individual's health status. One example type of time-series health features are sensor data from mobile health devices.

Clinical health features 206 are also illustrated in FIG. 2, which can include laboratory test values or other clinical measurements. These clinical health features 206 can be beneficial for diagnosing conditions, monitoring health status, and informing treatment decisions. They can be obtained from electronic health records, laboratory information systems, or directly from diagnostic equipment.

An input sequence of tokens in natural language embedding space 216 represents the transformation of the processed tabular health features 202, time-series health features 204, clinical health features 206, and additional prompt 214 into a format compatible with the sequence processing model 218. These tokens can be generated by a preprocessor (not shown in FIG. 2) and are designed to encapsulate the semantic and contextual information contained within the multi-modal health data and the query.

In particular, as examples, the tabular data 202 can be transformed into text tokens 208. The time-series data 204 can be processed by a time-series-encoder 210 to generate tokens in the natural language embedding space. Likewise, the clinical data 206 can be processed by a clinical data encoder 212 to generate tokens in the natural language embedding space.

The additional prompt 214, which is optional, can be a natural language query or instruction provided by a user or clinician. The additional prompt 214 can be specific to the individual's health status or a general inquiry related to health and wellness. It can be used to guide the focus of the health-related prediction or response generated by the system.

The sequence processing model 218, which is a type of neural network, receives the input sequence of tokens in natural language embedding space 216 and processes them to generate a health-related prediction or response. The sequence processing model 218 can be a large language model (LLM) that has been trained on diverse datasets, including professional examination data, patient-reported outcome data, and case studies validated by human experts. The model 218 can analyze the input sequence of tokens to infer patterns, correlations, and insights that inform the generation of the health-related prediction or response.

Finally, the response or output 220 is the output of the system's processing. This response or output 220 can take various forms, such as a natural language answer to the query, a health coaching message, a risk assessment, or a predictive user-submitted report. The response or output 220 is tailored to the individual's specific health data and the context of the query, ensuring that it is personalized and relevant. The response or output 220 can then be provided for presentation to the user or to a clinician treating the patient, thereby completing the system's cycle of generating health-related predictions and responses.

The system depicted in FIG. 2 can be employed in various implementations, such as a user-facing health and wellness chatbot, a clinician assistant software, or an early warning system in a medical treatment facility. The flexibility of the system allows for a wide range of applications in the healthcare domain, leveraging the capabilities of sequence processing models to provide personalized and contextually relevant health-related predictions and responses based on multi-modal health data.

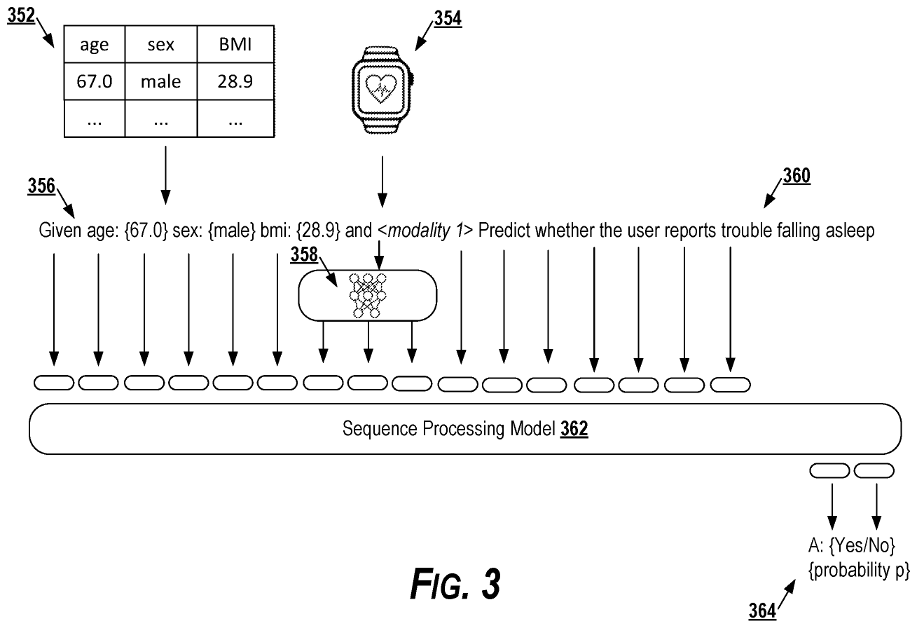

FIG. 3 illustrates a schematic representation of a system for generating health-related predictions based on multi-modal health data according to the present disclosure. The system, as depicted, can be implemented through a computing system comprising one or more computing devices configured to perform operations as described herein.

The system initiates with tabular health features 352, which, for example, can encompass demographic information. These features can be input into the system in a structured format, such as a table or a database record. The tabular health features 352 provide foundational data that can be beneficial in determining the context of subsequent health-related analyses and predictions.

Adjacent to the tabular health features 352 is mobile health data 354, which can represent dynamic health data collected over time. This may include, for example, heart rate data collected by a wearable device such as a smartwatch. The mobile health data 354 can be derived from a variety of sources, such as wearable fitness trackers or smartwatches, and can provide insight into the physiological aspects of an individual's health status.

In some implementations, the mobile health data 354 includes sensor data collected by a user computing device such as a smartwatch or a smartphone. This sensor data represents a variety of physiological metrics pertinent to the individual's health status. The data may be raw sensor outputs or may have been processed to derive additional health-related measures. For instance, a smartwatch may continuously monitor an individual's heart rate, generating a comprehensive dataset that is indicative of cardiovascular activity over time.

In some implementations, the mobile health data 354 may also include or be transformed to include textual data that provides context or additional information about the individual's health status. This textual data can be input by the user, derived from user interactions with the computing device, or generated by the device itself, such as textual interpretations of sensor data. For example, a user might enter notes regarding their dietary intake or symptoms experienced, which can be used alongside sensor data to enhance the personalization of health-related predictions.

In some implementations, the mobile health data 354 can include various health metrics that are derived from the sensor data collected by the user computing device. These metrics may include, but are not limited to, sleep-related scores and durations, such as light sleep, REM sleep, and deep sleep durations, as well as overall sleep efficiency and progress towards sleep goals. Additionally, the data may encompass various heart rate metrics, such as resting heart rate and RMSSD, along with respiratory rate and activity-related measures like fat-burn, cardio, and peak zone durations, TRIMP scores, and total steps taken. Each of these metrics provides insight into the individual's health and wellness, contributing to the comprehensive nature of the health profile utilized by the system.

In certain implementations, the mobile health data 354 may further include specific health indicators such as heart rate variability (HRV) measured by the root mean square of successive differences, respiratory rate, and resting heart rate. These indicators are particularly valuable for assessing an individual's autonomic nervous system activity and overall health condition. The system, leveraging the sequence processing model 362, utilizes these indicators to provide nuanced and medically relevant health-related predictions, as illustrated in FIG. 3. By incorporating such detailed physiological data, the system enhances its ability to deliver highly personalized and actionable health insights to users or healthcare professionals.

The tabular data 352 can be transformed into text tokens 356. The mobile health data 354 can be processed by a mobile health data encoder 358 to generate tokens in the natural language embedding space The mobile health data encoder 358 is a beneficial component that processes the mobile health data 354. The encoder 358 can transform the sensor data into a format that is amenable to analysis by the sequence processing model 362. This may include the transformation of raw sensor data into embeddings that can be understood within the context of the sequence processing model's framework.

In particular, in some implementations, prior to processing the mobile health data 354 with the sequence processing model 362, the system can perform a conversion step where sensor data is transformed into embeddings by a time-series encoder model 358. This transformation, as shown in FIG. 3, allows the system to represent the time-dependent characteristics of the sensor data in a format that is compatible with the sequence processing model's analytical framework. The embeddings capture the temporal dynamics and patterns within the sensor data, enabling the model 362 to more accurately interpret and utilize this information for generating health-related predictions.

An additional prompt 360 can be included in the system, which can be a natural language query or instruction provided by a user or clinician. The additional prompt 360 can be specific to the individual's health status or a general inquiry related to health and wellness. It can be used to guide the focus of the health-related prediction or response generated by the system.

The sequence processing model 362, which is a type of neural network, receives the input sequence of tokens in natural language embedding space and processes them to generate a health-related prediction or response. The sequence processing model 362 can be a large language model (LLM) that has been trained on diverse datasets, including professional examination data, patient-reported outcome data, and case studies validated by human experts. The model 362 can analyze the input sequence of tokens to infer patterns, correlations, and insights that inform the generation of the health-related prediction or response.

Finally, the response or output 364 is the output of the system's processing. This response or output 364 can take various forms, such as a natural language answer to the query, a health coaching message, a risk assessment, or a predictive user-submitted report. The response or output 364 is tailored to the individual's specific health data and the context of the query, ensuring that it is personalized and relevant. The response or output 364 can then be provided for presentation to the user or to a clinician treating the patient, thereby completing the system's cycle of generating health-related predictions and responses.

The system depicted in FIG. 3 can be employed in various implementations, such as a user-facing health and wellness chatbot, a clinician assistant software, or an early warning system in a medical treatment facility. The flexibility of the system allows for a wide range of applications in the healthcare domain, leveraging the capabilities of sequence processing models to provide personalized and contextually relevant health-related predictions and responses based on multi-modal health data.

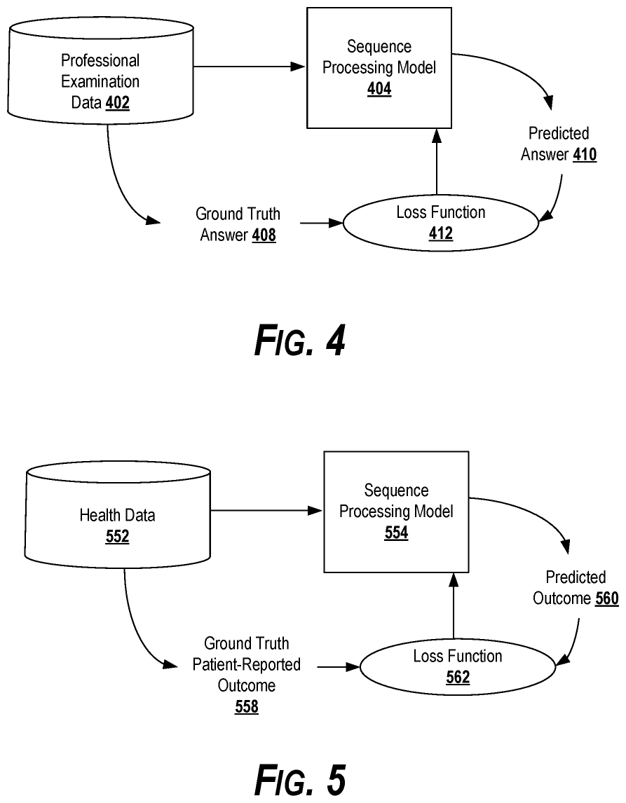

FIG. 4 illustrates a schematic representation of a system for generating health-related predictions and responses according to the present disclosure. This system can be implemented through a computing system comprising one or more computing devices configured to perform operations as described herein.

At the core of the system depicted in FIG. 4 is the sequence processing model 404, a type of neural network, which can include a large language model (LLM). The sequence processing model 404 can be trained to process and analyze multi-modal health data to generate personalized health-related predictions and responses when executed. This model 404 can receive input from various data sources, including professional examination data 402, and use this data to produce predicted answers 410 that are reflective of the individual's health status.

The professional examination data 402 represents a beneficial training input for the sequence processing model 404. This data can include a wide range of health-related information collected during professional medical examinations, such as patient history, physical examination findings, diagnostic test results, and clinical observations. The professional examination data 402 can be obtained from electronic health records, medical imaging systems, laboratory information systems, or directly from diagnostic equipment. This data provides a comprehensive view of an individual's health status, which is essential for the sequence processing model 404 to generate accurate and personalized health-related predictions.

The ground truth answer 408 serves as a benchmark for the predictions made by the sequence processing model 404. The ground truth answer 408 can be derived from expert analysis, clinical consensus, or empirical data, and represents the correct or expected outcome for a given health-related query or scenario (e.g., as included within the professional examination data 402). By comparing the predicted answer 410 generated by the sequence processing model 404 against the ground truth answer 408, the system can assess the accuracy and reliability of the model's predictions.

The predicted answer 410 is the output generated by the sequence processing model 404 based on the input professional examination data 402. The predicted answer 410 can take various forms, such as a natural language response, a numerical score, or a categorical classification. The predicted answer 410 is then evaluated against the ground truth answer 408 using the loss function 412, which quantifies the accuracy of the prediction. The results of this evaluation can be used to adjust the sequence processing model 404, refining its predictive capabilities for future queries.

The loss function 412 is an algorithmic component that measures the difference between the predicted answer 410 and the ground truth answer 408. The loss function 412 can be employed to evaluate the performance of the sequence processing model 404 and guide the optimization of the model's parameters during the training process. By minimizing the loss function 412, the system can improve the sequence processing model's ability to generate predictions that closely align with the ground truth, thereby enhancing the overall accuracy of the health-related predictions and responses.

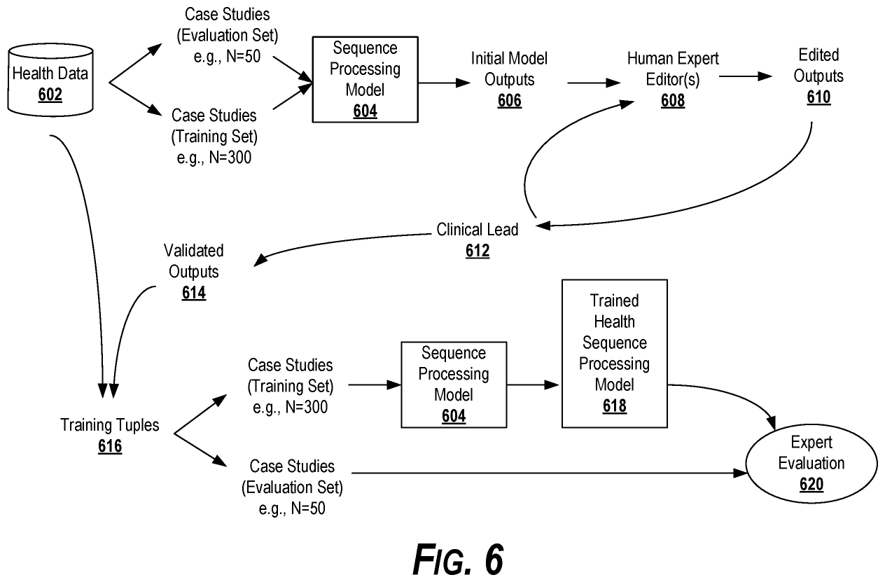

FIG. 5 illustrates a schematic representation of a system for generating health-related predictions and responses based on multi-modal health data according to the present disclosure. The system, as depicted, can be implemented through a computing system comprising one or more computing devices configured to perform operations as described herein.

The system initiates with health data 552, which can encompass a wide array of health-related information associated with an individual. This health data 552 can be sourced from various origins, such as electronic health records, patient self-reports, wearable device sensor outputs, or other health monitoring systems. The health data 552 can include, but is not limited to, time-series sensor data, tabular data, textual data, and clinical health features. This data provides a comprehensive overview of an individual's health status and can be beneficial for the accurate and personalized generation of health-related predictions.

At the center of the system is the sequence processing model 554, which is a type of neural network that can include a large language model (LLM). The sequence processing model 554 is trained to process and analyze the health data 552 to generate personalized health-related predictions and responses when executed. By leveraging the capabilities of the sequence processing model 554, the system can interpret complex multimodal health data and produce outputs that are tailored to the specific health profile of the individual.

The ground truth patient-reported outcome 558 serves as a standard or benchmark for evaluating the predictions made by the sequence processing model 554. This ground truth patient-reported outcome 558 can be obtained from direct patient feedback, clinical assessments, or validated health questionnaires. It represents the actual or expected health status or outcome for a particular health-related aspect or condition of the individual. By comparing the predicted outcome 560 generated by the sequence processing model 554 against the ground truth patient-reported outcome 558, the system can gauge the accuracy and relevance of the model's predictions.

The predicted outcome 560 is the result produced by the sequence processing model 554 after processing the health data 552. The predicted outcome 560 can be in various forms, such as a natural language explanation, a numerical score, or a categorical classification that aligns with health-related conditions or statuses. This predicted outcome 560 is reflective of the individual's health data and is intended to closely match the ground truth patient-reported outcome 558.

The loss function 562 is a algorithmic component within the system that quantifies the discrepancy between the predicted outcome 560 and the ground truth patient-reported outcome 558. The loss function 562 is utilized to assess the performance of the sequence processing model 554, providing a measure of the model's prediction error. By minimizing the loss through iterative training and optimization, the system can enhance the sequence processing model's 554 ability to generate more accurate and reliable health-related predictions and responses.

The system depicted in FIG. 5 demonstrates the present disclosure's approach to utilizing advanced machine learning techniques, such as the sequence processing model 554, to process health data 552 and generate personalized health-related predictions. By incorporating the ground truth patient-reported outcome 558 and employing the loss function 562 to refine the model's accuracy, the system ensures that the health-related predictions and responses are continually optimized for precision and personalization. This system can be employed in various healthcare applications, including personalized health monitoring, predictive health analytics, and patient-centered health interventions, thereby exemplifying the present disclosure's commitment to improving the quality and customization of health-related predictions and responses.

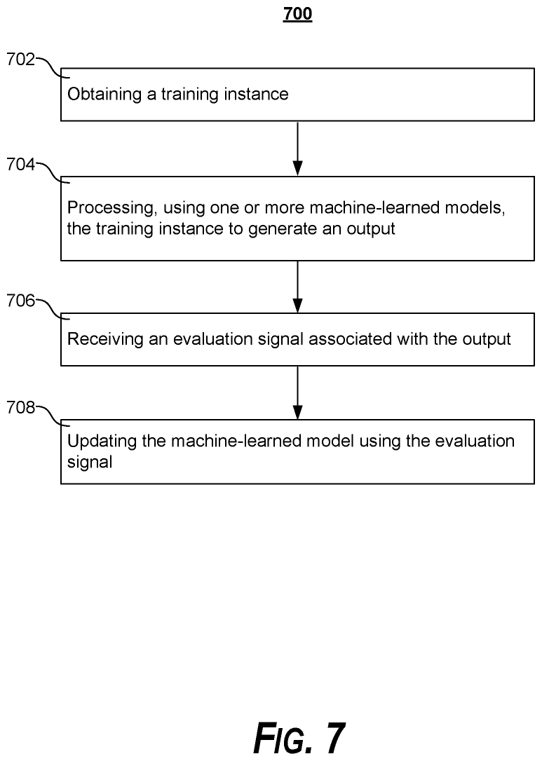

Referring now to FIG. 6, a schematic representation of a system for generating personalized health-related predictions and responses based on multi-modal health data according to the present disclosure is illustrated. The system can be implemented through a computing system comprising one or more computing devices configured to perform operations as described herein.

The system begins with health data 602, which can include a wide range of health-related information associated with an individual. Health data 602 can be obtained from various sources such as electronic health records, patient self-reports, wearable device sensor outputs, or other health monitoring systems. Health data 602 provides a comprehensive overview of an individual's health status and can be beneficial for the accurate and personalized generation of health-related predictions.

The sequence processing model 604 is a central component of the system and can be a type of neural network, including a large language model (LLM). The sequence processing model 604 can be trained to process and analyze health data 602 to generate personalized health-related predictions and responses when executed. By leveraging the capabilities of the sequence processing model 604, the system can interpret complex multimodal health data and produce outputs that are tailored to the specific health profile of the individual.

Initial model outputs 606 represent the preliminary health-related predictions and responses generated by the sequence processing model 604 based on the health data 602. Initial model outputs 606 can be in various forms, such as natural language explanations, numerical scores, or categorical classifications that align with health-related conditions or statuses. These outputs serve as a starting point for further refinement and validation.

Human expert editors 608 are involved in the process to review and edit the initial model outputs 606. These editors can be medical professionals or health data specialists who utilize their expertise to ensure that the health advice is not only accurate but also tailored to the specific considerations of individual health scenarios. The human expert editors 608 may edit the outputs to enhance their clarity, relevance, and adherence to current medical guidelines and best practices.

Edited outputs 610 are the result of the refinement process performed by human expert editors 608. Edited outputs 610 have undergone a review process to improve their accuracy and applicability to individual health cases. These edited outputs can be more precise and personalized than the initial model outputs 606, reflecting the expert knowledge and context-specific insights provided by the human expert editors 608.

A clinical lead 612 then conducts a thorough review of the edited outputs 610 to validate their medical accuracy and efficacy. The clinical lead 612 can be a highly experienced medical practitioner whose role is to confirm that the edited outputs meet the stringent standards required for medical advice. This validation step is beneficial to ensure that the outputs are of high quality and can be trusted by end-users.

Validated outputs 614 are the outputs that have been reviewed and approved by the clinical lead 612. These outputs are considered to be of high quality and are ready to be used as part of the training data for the sequence processing model 604. Validated outputs 614 represent expert-curated and clinically validated health-related predictions and responses that are optimized for accuracy and relevance.

Training tuples 616 are created by combining health data 602 with validated outputs 614. Each training tuple represents a complete case study, encompassing both the raw health data and the expert-validated responses. These training tuples are used to train the sequence processing model 604, ensuring that the model learns from high-quality, expert-curated information.

The trained health sequence processing model 618 is the enhanced version of the sequence processing model 604 after it has been trained on the training tuples 616. The trained health sequence processing model 618 has improved predictive accuracy and is more capable of providing personalized and contextually appropriate health coaching responses. This model can process new health data and generate predictions and responses that reflect the insights gained from the training process.

Finally, an expert evaluation process 620 can be performed. Expert evaluation 620 is a process in which the trained health sequence processing model 618 is assessed by experts to determine its performance and reliability. The experts can also evaluate the model's predictions and responses against new case studies or real-world health data to ensure that the model's outputs are consistent with expert knowledge and clinical standards.

The system depicted in FIG. 6 demonstrates the present disclosure's approach to integrating expert knowledge and machine learning techniques to produce personalized health-related predictions and responses. By leveraging the sequence processing model 604, training it with expert-curated data, and validating its outputs, the system can process complex health data and generate responses that are tailored to an individual's specific health status. This system can be employed in various healthcare applications, including diagnostic support tools, personalized health coaching systems, and early warning systems for patient care, thus exemplifying the present disclosure's commitment to enhancing the quality and personalization of health-related predictions and responses.

### Example Methods

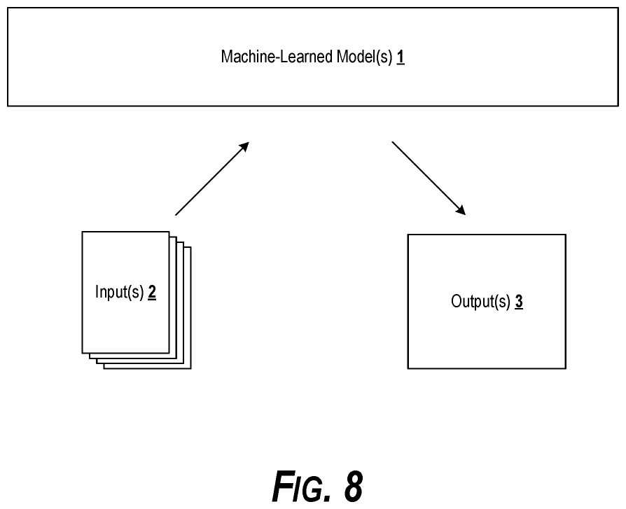

FIG. 7 depicts a flowchart of a method 700 for training one or more machine-learned models according to aspects of the present disclosure. For instance, an example machine-learned model can include a multi-modal sequence processing model.

One or more portion(s) of example method 700 can be implemented by a computing system that includes one or more computing devices such as, for example, computing systems described with reference to the other figures. Each respective portion of example method 700 can be performed by any (or any combination) of one or more computing devices. Moreover, one or more portion(s) of example method 700 can be implemented on the hardware components of the device(s) described herein, for example, to train one or more systems or models. FIG. 7 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 7 is described with reference to elements/terms described with respect to other systems and figures for exemplary illustrated purposes and is not meant to be limiting. One or more portions of example method 700 can be performed additionally, or alternatively, by other systems.

At 702, example method 700 can include obtaining a training instance. A set of training data can include a plurality of training instances divided between multiple datasets (e.g., a training dataset, a validation dataset, or testing dataset). A training instance can be labeled or unlabeled. Although referred to in example method 700 as a “training” instance, it is to be understood that runtime inferences can form training instances when a model is trained using an evaluation of the model's performance on that runtime instance (e.g., online training/learning). Example data types for the training instance and various tasks associated therewith are described throughout the present disclosure.

At 704, example method 700 can include processing, using one or more machine-learned models, the training instance to generate an output. The output can be directly obtained from the one or more machine-learned models or can be a downstream result of a chain of processing operations that includes an output of the one or more machine-learned models.

At 706, example method 700 can include receiving an evaluation signal associated with the output. The evaluation signal can be obtained using a loss function. Various determinations of loss can be used, such as mean squared error, likelihood loss, cross entropy loss, hinge loss, contrastive loss, or various other loss functions. The evaluation signal can be computed using known ground-truth labels (e.g., supervised learning), predicted or estimated labels (e.g., semi-or self-supervised learning), or without labels (e.g., unsupervised learning). The evaluation signal can be a reward (e.g., for reinforcement learning). The reward can be computed using a machine-learned reward model configured to generate rewards based on output(s) received. The reward can be computed using feedback data describing human feedback on the output(s).

At 708, example method 700 can include updating the machine-learned model using the evaluation signal. For example, values for parameters of the machine-learned model(s) can be learned, in some embodiments, using various training or learning techniques, such as, for example, backwards propagation. For example, the evaluation signal can be backpropagated from the output (or another source of the evaluation signal) through the machine-learned model(s) to update one or more parameters of the model(s) (e.g., based on a gradient of the evaluation signal with respect to the parameter value(s)). For example, system(s) containing one or more machine-learned models can be trained in an end-to-end manner. Gradient descent techniques can be used to iteratively update the parameters over a number of training iterations. In some implementations, performing backwards propagation of errors can include performing truncated backpropagation through time. Example method 700 can include implementing a number of generalization techniques (e.g., weight decays, dropouts, etc.) to improve the generalization capability of the models being trained.

In some implementations, example method 700 can be implemented for training a machine-learned model from an initialized state to a fully trained state (e.g., when the model exhibits a desired performance profile, such as based on accuracy, precision, recall, etc.).

In some implementations, example method 700 can be implemented for particular stages of a training procedure. For instance, in some implementations, example method 700 can be implemented for pre-training a machine-learned model. Pre-training can include, for instance, large-scale training over potentially noisy data to achieve a broad base of performance levels across a variety of tasks/data types. In some implementations, example method 700 can be implemented for fine-tuning a machine-learned model. Fine-tuning can include, for instance, smaller-scale training on higher-quality (e.g., labeled, curated, etc.) data. Fine-tuning can affect all or a portion of the parameters of a machine-learned model. For example, various portions of the machine-learned model can be “frozen” for certain training stages. For example, parameters associated with an embedding space can be “frozen” during fine-tuning (e.g., to retain information learned from a broader domain(s) than present in the fine-tuning dataset(s)). An example fine-tuning approach includes reinforcement learning. Reinforcement learning can be based on user feedback on model performance during use.

### Example Machine-Learned Models

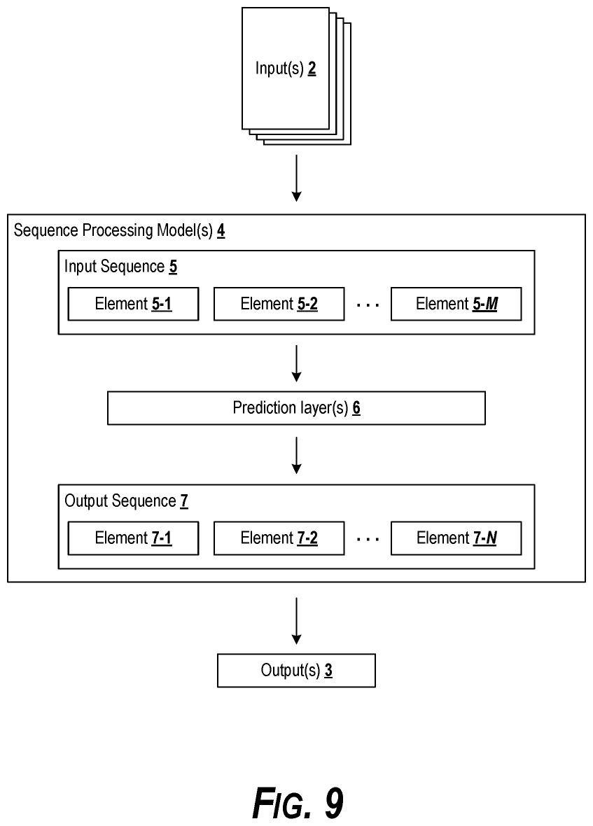

FIG. 8 is a block diagram of an example processing flow for using machine-learned model(s) 1 to process input(s) 2 to generate output(s) 3.

Machine-learned model(s) 1 can be or include one or multiple machine-learned models or model components. Example machine-learned models can include neural networks (e.g., deep neural networks). Example machine-learned models can include non-linear models or linear models. Example machine-learned models can use other architectures in lieu of or in addition to neural networks. Example machine-learned models can include decision tree based models, support vector machines, hidden Markov models, Bayesian networks, linear regression models, k-means clustering models, etc.

Example neural networks can include feed-forward neural networks, recurrent neural networks (RNNs), including long short-term memory (LSTM) based recurrent neural networks, convolutional neural networks (CNNs), diffusion models, generative-adversarial networks, or other forms of neural networks. Example neural networks can be deep neural networks. Some example machine-learned models can leverage an attention mechanism such as self-attention. For example, some example machine-learned models can include multi-headed self-attention models.

Machine-learned model(s) 1 can include a single or multiple instances of the same model configured to operate on data from input(s) 2. Machine-learned model(s) 1 can include an ensemble of different models that can cooperatively interact to process data from input(s) 2. For example, machine-learned model(s) 1 can employ a mixture-of-experts structure. See, e.g., Zhou et al., Mixture-of-Experts with Expert Choice Routing, ARXIV:2202.09368v2 (Oct. 14, 2022).

Input(s) 2 can generally include or otherwise represent various types of data. Input(s) 2 can include one type or many different types of data. Output(s) 3 can be data of the same type(s) or of different types of data as compared to input(s) 2. Output(s) 3 can include one type or many different types of data.

Example data types for input(s) 2 or output(s) 3 include natural language text data, software code data (e.g., source code, object code, machine code, or any other form of computer-readable instructions or programming languages), machine code data (e.g., binary code, assembly code, or other forms of machine-readable instructions that can be executed directly by a computer's central processing unit), assembly code data (e.g., low-level programming languages that use symbolic representations of machine code instructions to program a processing unit), genetic data or other chemical or biochemical data, image data, audio data, audiovisual data, haptic data, biometric data, medical data, financial data, statistical data, geographical data, astronomical data, historical data, sensor data generally (e.g., digital or analog values, such as voltage or other absolute or relative level measurement values from a real or artificial input, such as from an audio sensor, light sensor, displacement sensor, etc.), and the like. Data can be raw or processed and can be in any format or schema.

In multimodal inputs 2 or outputs 3, example combinations of data types include image data and audio data, image data and natural language data, natural language data and software code data, image data and biometric data, sensor data and medical data, etc. It is to be understood that any combination of data types in an input 2 or an output 3 can be present.

An example input 2 can include one or multiple data types, such as the example data types noted above. An example output 3 can include one or multiple data types, such as the example data types noted above. The data type(s) of input 2 can be the same as or different from the data type(s) of output 3. It is to be understood that the example data types noted above are provided for illustrative purposes only. Data types contemplated within the scope of the present disclosure are not limited to those examples noted above.

### Example Machine-Learned Sequence Processing Models

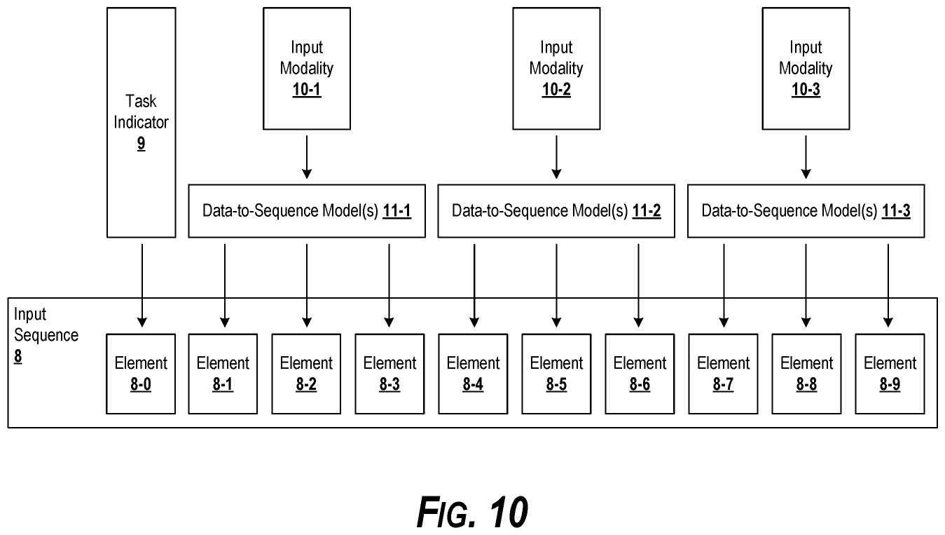

FIG. 9 is a block diagram of an example implementation of an example machine-learned model configured to process sequences of information. For instance, an example implementation of machine-learned model(s) 1 can include machine-learned sequence processing model(s) 4. An example system can pass input(s) 2 to sequence processing model(s) 4. Sequence processing model(s) 4 can include one or more machine-learned components. Sequence processing model(s) 4 can process the data from input(s) 2 to obtain an input sequence 5. Input sequence 5 can include one or more input elements 5-1, 5-2, . . . , 5-M, etc. obtained from input(s) 2. Sequence processing model 4 can process input sequence 5 using prediction layer(s) 6 to generate an output sequence 7. Output sequence 7 can include one or more output elements 7-1, 7-2, . . . , 7-N, etc. generated based on input sequence 5. The system can generate output(s) 3 based on output sequence 7.

Sequence processing model(s) 4 can include one or multiple machine-learned model components configured to ingest, generate, or otherwise reason over sequences of information. For example, some example sequence processing models in the text domain are referred to as “Large Language Models,” or LLMs. See, e.g., PaLM 2 Technical Report, GOOGLE, https://ai.google/static/documents/palm2techreport.pdf (n.d.). Other example sequence processing models can operate in other domains, such as image domains, see, e.g., Dosovitskiy et al., An Image is Worth 16×16 Words: Transformers for Image Recognition at Scale, ARXIV:2010.11929v2 (Jun. 3, 2021), audio domains, see, e.g., Agostinelli et al., MusicLM: Generating Music From Text, ARXIV:2301.11325v1 (Jan. 26, 2023), biochemical domains, see, e.g., Jumper et al., Highly accurate protein structure prediction with AlphaFold, 596 Nature 583 (Aug. 26, 2021), by way of example. Sequence processing model(s) 4 can process one or multiple types of data simultaneously. Sequence processing model(s) 4 can include relatively large models (e.g., more parameters, computationally expensive, etc.), relatively small models (e.g., fewer parameters, computationally lightweight, etc.), or both.

In general, sequence processing model(s) 4 can obtain input sequence 5 using data from input(s) 2. For instance, input sequence 5 can include a representation of data from input(s) 2 in a format understood by sequence processing model(s) 4. One or more machine-learned components of sequence processing model(s) 4 can ingest the data from input(s) 2, parse the data into pieces compatible with the processing architectures of sequence processing model(s) 4 (e.g., via “tokenization”), and project the pieces into an input space associated with prediction layer(s) 6 (e.g., via “embedding”).

Sequence processing model(s) 4 can ingest the data from input(s) 2 and parse the data into a sequence of elements to obtain input sequence 5. For example, a portion of input data from input(s) 2 can be broken down into pieces that collectively represent the content of the portion of the input data. The pieces can provide the elements of the sequence.

Elements 5-1, 5-2, . . . , 5-M can represent, in some cases, building blocks for capturing or expressing meaningful information in a particular data domain. For instance, the elements can describe “atomic units” across one or more domains. For example, for textual input source(s), the elements can correspond to groups of one or more words or sub-word components, such as sets of one or more characters.

For example, elements 5-1, 5-2, . . . , 5-M can represent tokens obtained using a tokenizer. For instance, a tokenizer can process a given portion of an input source and output a series of tokens (e.g., corresponding to input elements 5-1, 5-2, . . . , 5-M) that represent the portion of the input source. Various approaches to tokenization can be used. For instance, textual input source(s) can be tokenized using a byte-pair encoding (BPE) technique. See, e.g., Kudo et al., SentencePiece: A simple and language independent subword tokenizer and detokenizer for Neural Text Processing, PROCEEDINGS OF THE 2018 CONFERENCE ON EMPIRICAL METHODS IN NATURAL LANGUAGE PROCESSING (System Demonstrations), pages 66-71 (Oct. 31-Nov. 4, 2018), https://aclanthology.org/D18-2012.pdf. Image-based input source(s) can be tokenized by extracting and serializing patches from an image.

In general, arbitrary data types can be serialized and processed into input sequence 5. It is to be understood that element(s) 5-1, 5-2, . . . , 5-M depicted in FIG. 9 can be the tokens or can be the embedded representations thereof.

Prediction layer(s) 6 can predict one or more output elements 7-1, 7-2, . . . , 7-N based on the input elements. Prediction layer(s) 6 can include one or more machine-learned model architectures, such as one or more layers of learned parameters that manipulate and transform the input(s) to extract higher-order meaning from, and relationships between, input element(s) 5-1, 5-2, . . . , 5-M. In this manner, for instance, example prediction layer(s) 6 can predict new output element(s) in view of the context provided by input sequence 5.

Prediction layer(s) 6 can evaluate associations between portions of input sequence 5 and a particular output element. These associations can inform a prediction of the likelihood that a particular output follows the input context. For example, consider the textual snippet, “The carpenter's toolbox was small and heavy. It was full of ______. ” Example prediction layer(s) 6 can identify that “It” refers back to “toolbox” by determining a relationship between the respective embeddings. Example prediction layer(s) 6 can also link “It” to the attributes of the toolbox, such as “small” and “heavy.” Based on these associations, prediction layer(s) 6 can, for instance, assign a higher probability to the word “nails” than to the word “sawdust.”A transformer is an example architecture that can be used in prediction layer(s) 4. See, e.g., Vaswani et al., Attention Is All You Need, ARXIV:1706.03762v7 (Aug. 2, 2023). A transformer is an example of a machine-learned model architecture that uses an attention mechanism to compute associations between items within a context window. The context window can include a sequence that contains input sequence 5 and potentially one or more output element(s) 7-1, 7-2, . . . , 7-N. A transformer block can include one or more attention layer(s) and one or more post-attention layer(s) (e.g., feedforward layer(s), such as a multi-layer perceptron).

Prediction layer(s) 6 can include other machine-learned model architectures in addition to or in lieu of transformer-based architectures. For example, recurrent neural networks (RNNs) and long short-term memory (LSTM) models can also be used, as well as convolutional neural networks (CNNs). In general, prediction layer(s) 6 can leverage various kinds of artificial neural networks that can understand or generate sequences of information.

Output sequence 7 can include or otherwise represent the same or different data types as input sequence 5. For instance, input sequence 5 can represent textual data, and output sequence 7 can represent textual data. Input sequence 5 can represent image, audio, or audiovisual data, and output sequence 7 can represent textual data (e.g., describing the image, audio, or audiovisual data). It is to be understood that prediction layer(s) 6, and any other interstitial model components of sequence processing model(s) 4, can be configured to receive a variety of data types in input sequence(s) 5 and output a variety of data types in output sequence(s) 7.

Output sequence 7 can have various relationships to input sequence 5. Output sequence 7 can be a continuation of input sequence 5. Output sequence 7 can be complementary to input sequence 5. Output sequence 7 can translate, transform, augment, or otherwise modify input sequence 5. Output sequence 7 can answer, evaluate, confirm, or otherwise respond to input sequence 5. Output sequence 7 can implement (or describe instructions for implementing) an instruction provided via input sequence 5.

Output sequence 7 can be generated autoregressively. For instance, for some applications, an output of one or more prediction layer(s) 6 can be passed through one or more output layers (e.g., softmax layer) to obtain a probability distribution over an output vocabulary (e.g., a textual or symbolic vocabulary) conditioned on a set of input elements in a context window. In this manner, for instance, output sequence 7 can be autoregressively generated by sampling a likely next output element, adding that element to the context window, and re-generating the probability distribution based on the updated context window, and sampling a likely next output element, and so forth.

Output sequence 7 can also be generated non-autoregressively. For instance, multiple output elements of output sequence 7 can be predicted together without explicit sequential conditioning on each other. See, e.g., Saharia et al., Non-Autoregressive Machine Translation with Latent Alignments, ARXIV:2004.07437v3 (Nov. 16, 2020).

Output sequence 7 can include one or multiple portions or elements. In an example content generation configuration, output sequence 7 can include multiple elements corresponding to multiple portions of a generated output sequence (e.g., a textual sentence, values of a discretized waveform, computer code, etc.). In an example classification configuration, output sequence 7 can include a single element associated with a classification output. For instance, an output “vocabulary” can include a set of classes into which an input sequence is to be classified. For instance, a vision transformer block can pass latent state information to a multilayer perceptron that outputs a likely class value associated with an input image.

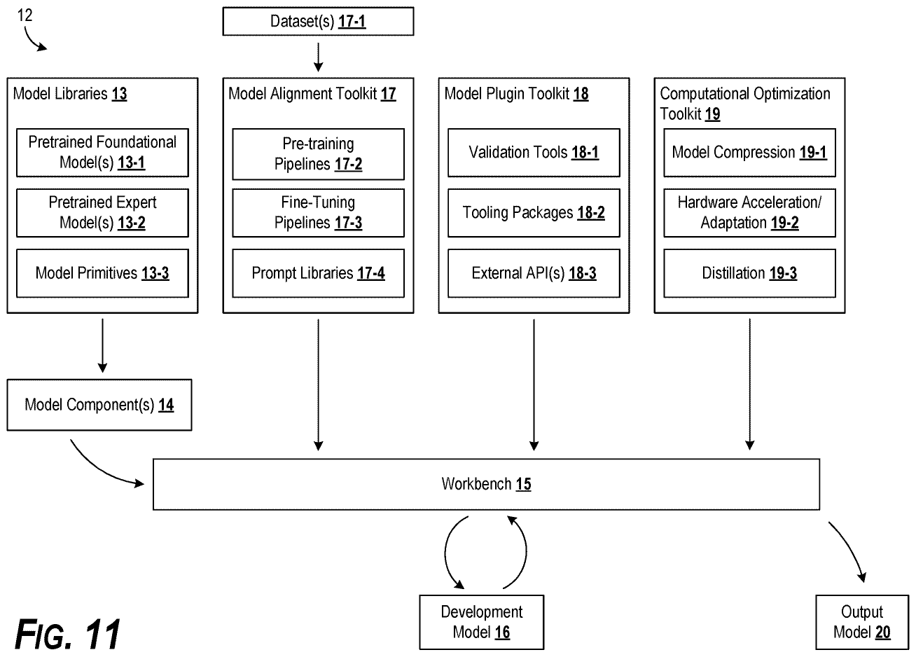

FIG. 10 is a block diagram of an example technique for populating an example input sequence 8. Input sequence 8 can include various functional elements that form part of the model infrastructure, such as an element 8-0 obtained from a task indicator 9 that signals to any model(s) that process input sequence 8 that a particular task is being performed (e.g., to help adapt a performance of the model(s) to that particular task). Input sequence 8 can include various data elements from different data modalities. For instance, an input modality 10-1 can include one modality of data. A data-to-sequence model 11-1 can process data from input modality 10-1 to project the data into a format compatible with input sequence 8 (e.g., one or more vectors dimensioned according to the dimensions of input sequence 8) to obtain elements 8-1, 8-2, 8-3. Another input modality 10-2 can include a different modality of data. A data-to-sequence model 11-2 can project data from input modality 10-2 into a format compatible with input sequence 8 to obtain elements 8-4, 8-5, 8-6. Another input modality 10-3 can include yet another different modality of data. A data-to-sequence model 11-3 can project data from input modality 10-3 into a format compatible with input sequence 8 to obtain elements 8-7, 8-8, 8-9.

Input sequence 8 can be the same as or different from input sequence 5. Input sequence 8 can be a multimodal input sequence that contains elements that represent data from different modalities using a common dimensional representation. For instance, an embedding space can have P dimensions. Input sequence 8 can be configured to contain a plurality of elements that have P dimensions. In this manner, for instance, example implementations can facilitate information extraction and reasoning across diverse data modalities by projecting data into elements in the same embedding space for comparison, combination, or other computations therebetween.

For example, elements 8-0, . . . , 8-9 can indicate particular locations within a multidimensional embedding space. Some elements can map to a set of discrete locations in the embedding space. For instance, elements that correspond to discrete members of a predetermined vocabulary of tokens can map to discrete locations in the embedding space that are associated with those tokens. Other elements can be continuously distributed across the embedding space. For instance, some data types can be broken down into continuously defined portions (e.g., image patches) that can be described using continuously distributed locations within the embedding space.

In some implementations, the expressive power of the embedding space may not be limited to meanings associated with any particular set of tokens or other building blocks. For example, a continuous embedding space can encode a spectrum of high-order information. An individual piece of information (e.g., a token) can map to a particular point in that space: for instance, a token for the word “dog” can be projected to an embedded value that points to a particular location in the embedding space associated with canine-related information. Similarly, an image patch of an image of a dog on grass can also be projected into the embedding space. In some implementations, the projection of the image of the dog can be similar to the projection of the word “dog” while also having similarity to a projection of the word “grass,” while potentially being different from both. In some implementations, the projection of the image patch may not exactly align with any single projection of a single word. In some implementations, the projection of the image patch can align with a combination of the projections of the words “dog” and “grass.” In this manner, for instance, a high-order embedding space can encode information that can be independent of data modalities in which the information is expressed.

Task indicator 9 can include a model or model component configured to identify a task being performed and inject, into input sequence 8, an input value represented by element 8-0 that signals which task is being performed. For instance, the input value can be provided as a data type associated with an input modality and projected along with that input modality (e.g., the input value can be a textual task label that is embedded along with other textual data in the input; the input value can be a pixel-based representation of a task that is embedded along with other image data in the input; etc.). The input value can be provided as a data type that differs from or is at least independent from other input(s). For instance, the input value represented by element 8-0 can be a learned within a continuous embedding space.

Input modalities 10-1, 10-2, and 10-3 can be associated with various different data types (e.g., as described above with respect to input(s) 2 and output(s) 3).

Data-to-sequence models 11-1, 11-2, and 11-3 can be the same or different from each other. Data-to-sequence models 11-1, 11-2, and 11-3 can be adapted to each respective input modality 10-1, 10-2, and 10-3. For example, a textual data-to-sequence model can subdivide a portion of input text and project the subdivisions into element(s) in input sequence 8 (e.g., elements 8-1, 8-2, 8-3, etc.). An image data-to-sequence model can subdivide an input image and project the subdivisions into element(s) in input sequence 8 (e.g., elements 8-4, 8-5, 8-6, etc.). An arbitrary datatype data-to-sequence model can subdivide an input of that arbitrary datatype and project the subdivisions into element(s) in input sequence 8 (e.g., elements 8-7, 8-8, 8-9, etc.).

Data-to-sequence models 11-1, 11-2, and 11-3 can form part of machine-learned sequence processing model(s) 4. Data-to-sequence models 11-1, 11-2, and 11-3 can be jointly trained with or trained independently from machine-learned sequence processing model(s) 4. Data-to-sequence models 11-1, 11-2, and 11-3 can be trained end-to-end with machine-learned sequence processing model(s) 4.

### Example Machine-Learned Model Development Platform

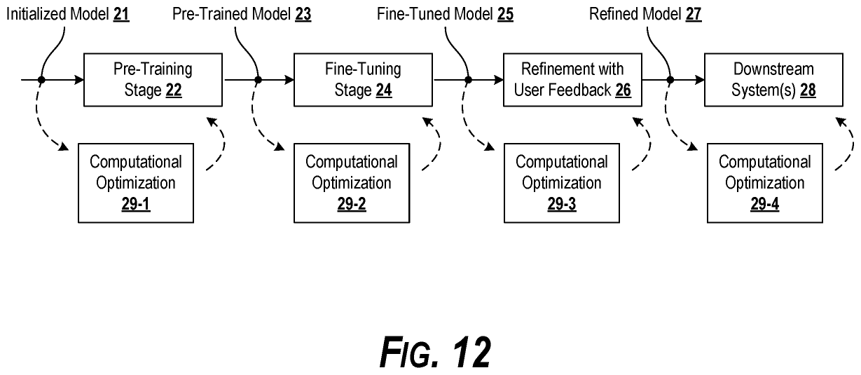

FIG. 11 is a block diagram of an example model development platform 12 that can facilitate creation, adaptation, and refinement of example machine-learned models (e.g., machine-learned model(s) 1, sequence processing model(s) 4, etc.). Model development platform 12 can provide a number of different toolkits that developer systems can employ in the development of new or adapted machine-learned models.

Model development platform 12 can provide one or more model libraries 13 containing building blocks for new models. Model libraries 13 can include one or more pre-trained foundational models 13-1, which can provide a backbone of processing power across various tasks. Model libraries 13 can include one or more pre-trained expert models 13-2, which can be focused on performance in particular domains of expertise. Model libraries 13 can include various model primitives 13-3, which can provide low-level architectures or components (optionally pre-trained), which can be assembled in various arrangements as desired.

Model development platform 12 can receive selections of various model components 14. Model development platform 12 can pass selected model components 14 to a workbench 15 that combines selected model components 14 into a development model 16.

Workbench 15 can facilitate further refinement and adaptation of development model 16 by leveraging a number of different toolkits integrated with model development platform 12. For example, workbench 15 can facilitate alignment of the development model 16 with a desired performance profile on various tasks using a model alignment toolkit 17.

Model alignment toolkit 17 can provide a number of tools for causing development model 16 to generate outputs aligned with desired behavioral characteristics.

Alignment can include increasing an accuracy, precision, recall, etc. of model outputs.

Alignment can include enforcing output styles, schema, or other preferential characteristics of model outputs. Alignment can be general or domain-specific. For instance, a pre-trained foundational model 13-1 can begin with an initial level of performance across multiple domains. Alignment of the pre-trained foundational model 13-1 can include improving a performance in a particular domain of information or tasks (e.g., even at the expense of performance in another domain of information or tasks).

Model alignment toolkit 17 can integrate one or more dataset(s) 17-1 for aligning development model 16. Curated dataset(s) 17-1 can include labeled or unlabeled training data. Dataset(s) 17-1 can be obtained from public domain datasets. Dataset(s) 17-1 can be obtained from private datasets associated with one or more developer system(s) for the alignment of bespoke machine-learned model(s) customized for private use-cases.

Pre-training pipelines 17-2 can include a machine-learned model training workflow configured to update development model 16 over large-scale, potentially noisy datasets. For example, pre-training can leverage unsupervised learning techniques (e.g., de-noising, etc.) to process large numbers of training instances to update model parameters from an initialized state and achieve a desired baseline performance. Pre-training pipelines 17-2 can leverage unlabeled datasets in dataset(s) 17-1 to perform pre-training. Workbench 15 can implement a pre-training pipeline 17-2 to pre-train development model 16.

Fine-tuning pipelines 17-3 can include a machine-learned model training workflow configured to refine the model parameters of development model 16 with higher-quality data. Fine-tuning pipelines 17-3 can update development model 16 by conducting supervised training with labeled dataset(s) in dataset(s) 17-1. Fine-tuning pipelines 17-3 can update development model 16 by conducting reinforcement learning using reward signals from user feedback signals. Workbench 15 can implement a fine-tuning pipeline 17-3 to fine-tune development model 16.

Prompt libraries 17-4 can include sets of inputs configured to induce behavior aligned with desired performance criteria. Prompt libraries 17-4 can include few-shot prompts (e.g., inputs providing examples of desired model outputs for prepending to a desired runtime query), chain-of-thought prompts (e.g., inputs providing step-by-step reasoning within the exemplars to facilitate thorough reasoning by the model), and the like.

Example prompts can be retrieved from an available repository of prompt libraries 17-4. Example prompts can be contributed by one or more developer systems using workbench 15.

In some implementations, pre-trained or fine-tuned models can achieve satisfactory performance without exemplars in the inputs. For instance, zero-shot prompts can include inputs that lack exemplars. Zero-shot prompts can be within a domain within a training dataset or outside of the training domain(s).

Prompt libraries 17-4 can include one or more prompt engineering tools. Prompt engineering tools can provide workflows for retrieving or learning optimized prompt values. Prompt engineering tools can facilitate directly learning prompt values (e.g., input element values) based one or more training iterations. Workbench 15 can implement prompt engineering tools in development model 16.

Prompt libraries 17-4 can include pipelines for prompt generation. For example, inputs can be generated using development model 16 itself or other machine-learned models. In this manner, for instance, a first model can process information about a task and output a input for a second model to process in order to perform a step of the task.

The second model can be the same as or different from the first model. Workbench 15 can implement prompt generation pipelines in development model 16.

Prompt libraries 17-4 can include pipelines for context injection. For instance, a performance of development model 16 on a particular task can improve if provided with additional context for performing the task. Prompt libraries 17-4 can include software components configured to identify desired context, retrieve the context from an external source (e.g., a database, a sensor, etc.), and add the context to the input prompt. Workbench 15 can implement context injection pipelines in development model 16.

Although various training examples described herein with respect to model development platform 12 refer to “pre-training” and “fine-tuning,” it is to be understood that model alignment toolkit 17 can generally support a wide variety of training techniques adapted for training a wide variety of machine-learned models. Example training techniques can correspond to the example training method 700 described above.

Model development platform 12 can include a model plugin toolkit 18. Model plugin toolkit 18 can include a variety of tools configured for augmenting the functionality of a machine-learned model by integrating the machine-learned model with other systems, devices, and software components. For instance, a machine-learned model can use tools to increase performance quality where appropriate. For instance, deterministic tasks can be offloaded to dedicated tools in lieu of probabilistically performing the task with an increased risk of error. For instance, instead of autoregressively predicting the solution to a system of equations, a machine-learned model can recognize a tool to call for obtaining the solution and pass the system of equations to the appropriate tool. The tool can be a traditional system of equations solver that can operate deterministically to resolve the system of equations. The output of the tool can be returned in response to the original query. In this manner, tool use can allow some example models to focus on the strengths of machine-learned models—e.g., understanding an intent in an unstructured request for a task—while augmenting the performance of the model by offloading certain tasks to a more focused tool for rote application of deterministic algorithms to a well-defined problem.

Model plugin toolkit 18 can include validation tools 18-1. Validation tools 18-1 can include tools that can parse and confirm output(s) of a machine-learned model. Validation tools 18-1 can include engineered heuristics that establish certain thresholds applied to model outputs. For example, validation tools 18-1 can ground the outputs of machine-learned models to structured data sources (e.g., to mitigate “hallucinations”).

Model plugin toolkit 18 can include tooling packages 18-2 for implementing one or more tools that can include scripts or other executable code that can be executed alongside development model 16. Tooling packages 18-2 can include one or more inputs configured to cause machine-learned model(s) to implement the tools (e.g., few-shot prompts that induce a model to output tool calls in the proper syntax, etc.). Tooling packages 18-2 can include, for instance, fine-tuning training data for training a model to use a tool.

Model plugin toolkit 18 can include interfaces for calling external application programming interfaces (APIs) 18-3. For instance, in addition to or in lieu of implementing tool calls or tool code directly with development model 16, development model 16 can be aligned to output instruction that initiate API calls to send or obtain data via external systems.

Model plugin toolkit 18 can integrate with prompt libraries 17-4 to build a catalog of available tools for use with development model 16. For instance, a model can receive, in an input, a catalog of available tools, and the model can generate an output that selects a tool from the available tools and initiates a tool call for using the tool.

Model development platform 12 can include a computational optimization toolkit 19 for optimizing a computational performance of development model 16. For instance, tools for model compression 19-1 can allow development model 16 to be reduced in size while maintaining a desired level of performance. For instance, model compression 19-1 can include quantization workflows, weight pruning and sparsification techniques, etc. Tools for hardware acceleration 19-2 can facilitate the configuration of the model storage and execution formats to operate optimally on different hardware resources. For instance, hardware acceleration 19-2 can include tools for optimally sharding models for distributed processing over multiple processing units for increased bandwidth, lower unified memory requirements, etc. Tools for distillation 19-3 can provide for the training of lighter-weight models based on the knowledge encoded in development model 16. For instance, development model 16 can be a highly performant, large machine-learned model optimized using model development platform 12. To obtain a lightweight model for running in resource-constrained environments, a smaller model can be a “student model” that learns to imitate development model 16 as a “teacher model.” In this manner, for instance, the investment in learning the parameters and configurations of development model 16 can be efficiently transferred to a smaller model for more efficient inference.

Workbench 15 can implement one, multiple, or none of the toolkits implemented in model development platform 12. Workbench 15 can output an output model 20 based on development model 16. Output model 20 can be a deployment version of development model 16. Output model 20 can be a development or training checkpoint of development model 16. Output model 20 can be a distilled, compressed, or otherwise optimized version of development model 16.

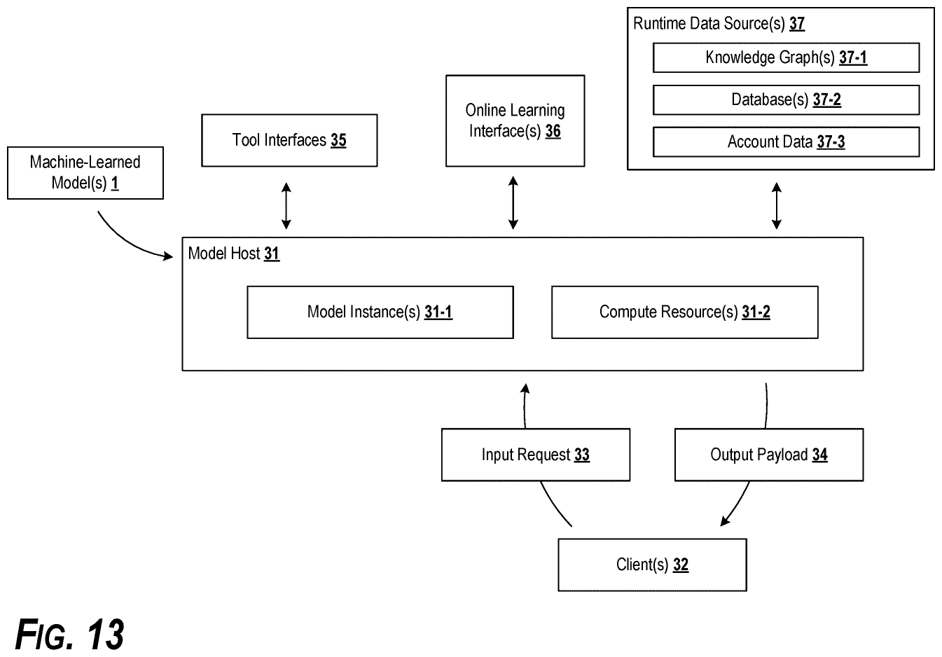

FIG. 12 is a block diagram of an example training flow for training a machine-learned development model 16. One or more portion(s) of the example training flow can be implemented by a computing system that includes one or more computing devices such as, for example, computing systems described with reference to the other figures. Each respective portion of the example training flow can be performed by any (or any combination) of one or more computing devices. Moreover, one or more portion(s) of the example training flow can be implemented on the hardware components of the device(s) described herein, for example, to train one or more systems or models. FIG. 12 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, or modified in various ways without deviating from the scope of the present disclosure. FIG. 12 is described with reference to elements/terms described with respect to other systems and figures for exemplary illustrated purposes and is not meant to be limiting. One or more portions of the example training flow can be performed additionally, or alternatively, by other systems.

Initially, development model 16 can persist in an initial state as an initialized model 21. Development model 16 can be initialized with weight values. Initial weight values can be random or based on an initialization schema. Initial weight values can be based on prior pre-training for the same or for a different model.

Initialized model 21 can undergo pre-training in a pre-training stage 22. Pre-training stage 22 can be implemented using one or more pre-training pipelines 17-2 over data from dataset(s) 17-1. Pre-training can be omitted, for example, if initialized model 21 is already pre-trained (e.g., development model 16 contains, is, or is based on a pre-trained foundational model or an expert model).

Pre-trained model 23 can then be a new version of development model 16, which can persist as development model 16 or as a new development model. Pre-trained model 23 can be the initial state if development model 16 was already pre-trained. Pre-trained model 23 can undergo fine-tuning in a fine-tuning stage 24. Fine-tuning stage 24 can be implemented using one or more fine-tuning pipelines 17-3 over data from dataset(s) 17-1. Fine-tuning can be omitted, for example, if a pre-trained model as satisfactory performance, if the model was already fine-tuned, or if other tuning approaches are preferred.

Fine-tuned model 29 can then be a new version of development model 16, which can persist as development model 16 or as a new development model. Fine-tuned model 29 can be the initial state if development model 16 was already fine-tuned. Fine-tuned model 29 can undergo refinement with user feedback 26. For instance, refinement with user feedback 26 can include reinforcement learning, optionally based on human feedback from human users of fine-tuned model 25. As reinforcement learning can be a form of fine-tuning, it is to be understood that fine-tuning stage 24 can subsume the stage for refining with user feedback 26. Refinement with user feedback 26 can produce a refined model 27. Refined model 27 can be output to downstream system(s) 28 for deployment or further development.

In some implementations, computational optimization operations can be applied before, during, or after each stage. For instance, initialized model 21 can undergo computational optimization 29-1 (e.g., using computational optimization toolkit 19) before pre-training stage 22. Pre-trained model 23 can undergo computational optimization 29-2 (e.g., using computational optimization toolkit 19) before fine-tuning stage 24. Fine-tuned model 25 can undergo computational optimization 29-3 (e.g., using computational optimization toolkit 19) before refinement with user feedback 26. Refined model 27 can undergo computational optimization 29-4 (e.g., using computational optimization toolkit 19) before output to downstream system(s) 28. Computational optimization(s) 29-1, . . . , 29-4 can all be the same, all be different, or include at least some different optimization techniques.

### Example Machine-Learned Model Inference System

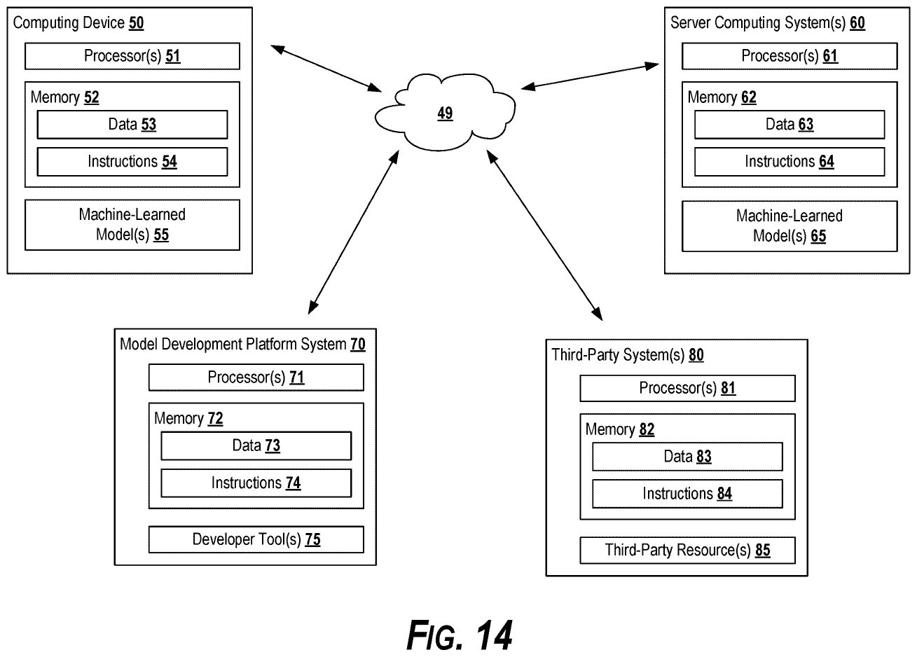

FIG. 13 is a block diagram of an inference system for operating one or more machine-learned model(s) 1 to perform inference (e.g., for training, for deployment, etc.). A model host 31 can receive machine-learned model(s) 1. Model host 31 can host one or more model instance(s) 31-1, which can be one or multiple instances of one or multiple models. Model host 31 can host model instance(s) 31-1 using available compute resources 31-2 associated with model host 31.

Model host 31 can perform inference on behalf of one or more client(s) 32. Client(s) 32 can transmit an input request 33 to model host 31. Using input request 33, model host 31 can obtain input(s) 2 for input to machine-learned model(s) 1. Machine-learned model(s) 1 can process input(s) 2 to generate output(s) 3. Using output(s) 3, model host 31 can return an output payload 34 for responding to input request 33 from client(s) 32. Output payload 34 can include or be based on output(s) 3.

Model host 31 can leverage various other resources and tools to augment the inference task. For instance, model host 31 can communicate with tool interfaces 35 to facilitate tool use by model instance(s) 31-1. Tool interfaces 35 can include local or remote APIs. Tool interfaces 35 can include integrated scripts or other software functionality. Model host 31 can engage online learning interface(s) 36 to facilitate ongoing improvements to machine-learned model(s) 1. For instance, online learning interface(s) 36 can be used within reinforcement learning loops to retrieve user feedback on inferences served by model host 31. Model host 31 can access runtime data source(s) 37 for augmenting input(s) 2 with additional contextual information. For instance, runtime data source(s) 37 can include a knowledge graph 37-1 that facilitates structured information retrieval for information associated with input request(s) 33 (e.g., a search engine service). Runtime data source(s) 37 can include public or private, external or local database(s) 37-2 that can store information associated with input request(s) 33 for augmenting input(s) 2. Runtime data source(s) 37 can include account data 37-3 which can be retrieved in association with a user account corresponding to a client 32 for customizing the behavior of model host 31 accordingly.

Model host 31 can be implemented by one or multiple computing devices or systems. Client(s) 2 can be implemented by one or multiple computing devices or systems, which can include computing devices or systems shared with model host 31.

For example, model host 31 can operate on a server system that provides a machine-learning service to client device(s) that operate client(s) 32 (e.g., over a local or wide-area network). Client device(s) can be end-user devices used by individuals. Client device(s) can be server systems that operate client(s) 32 to provide various functionality as a service to downstream end-user devices.

In some implementations, model host 31 can operate on a same device or system as client(s) 32. Model host 31 can be a machine-learning service that runs on-device to provide machine-learning functionality to one or multiple applications operating on a client device, which can include an application implementing client(s) 32. Model host 31 can be a part of a same application as client(s) 32. For instance, model host 31 can be a subroutine or method implemented by one part of an application, and client(s) 32 can be another subroutine or method that engages model host 31 to perform inference functions within the application. It is to be understood that model host 31 and client(s) 32 can have various different configurations.

Model instance(s) 31-1 can include one or more machine-learned models that are available for performing inference. Model instance(s) 31-1 can include weights or other model components that are stored on in persistent storage, temporarily cached, or loaded into high-speed memory. Model instance(s) 31-1 can include multiple instance(s) of the same model (e.g., for parallel execution of more requests on the same model). Model instance(s) 31-1 can include instance(s) of different model(s). Model instance(s) 31-1 can include cached intermediate states of active or inactive model(s) used to accelerate inference of those models. For instance, an inference session with a particular model may generate significant amounts of computational results that can be re-used for future inference runs (e.g., using a KV cache for transformer-based models). These computational results can be saved in association with that inference session so that session can be executed more efficiently when resumed.

Compute resource(s) 31-2 can include one or more processors (central processing units, graphical processing units, tensor processing units, machine-learning accelerators, etc.) connected to one or more memory devices. Compute resource(s) 31-2 can include a dynamic pool of available resources shared with other processes. Compute resource(s) 31-2 can include memory devices large enough to fit an entire model instance in a single memory instance. Compute resource(s) 31-2 can also shard model instance(s) across multiple memory devices (e.g., using data parallelization or tensor parallelization, etc.). This can be done to increase parallelization or to execute a large model using multiple memory devices which individually might not be able to fit the entire model into memory.

Input request 33 can include data for input(s) 2. Model host 31 can process input request 33 to obtain input(s) 2. Input(s) 2 can be obtained directly from input request 33 or can be retrieved using input request 33. Input request 33 can be submitted to model host 31 via an API.

Model host 31 can perform inference over batches of input requests 33 in parallel. For instance, a model instance 31-1 can be configured with an input structure that has a batch dimension. Separate input(s) 2 can be distributed across the batch dimension (e.g., rows of an array). The separate input(s) 2 can include completely different contexts. The separate input(s) 2 can be multiple inference steps of the same task. The separate input(s) 2 can be staggered in an input structure, such that any given inference cycle can be operating on different portions of the respective input(s) 2. In this manner, for instance, model host 31 can perform inference on the batch in parallel, such that output(s) 3 can also contain the batch dimension and return the inference results for the batched input(s) 2 in parallel. In this manner, for instance, batches of input request(s) 33 can be processed in parallel for higher throughput of output payload(s) 34.

Output payload 34 can include or be based on output(s) 3 from machine-learned model(s) 1. Model host 31 can process output(s) 3 to obtain output payload 34. This can include chaining multiple rounds of inference (e.g., iteratively, recursively, across the same model(s) or different model(s)) to arrive at a final output for a task to be returned in output payload 34. Output payload 34 can be transmitted to client(s) 32 via an API.

Online learning interface(s) 36 can facilitate reinforcement learning of machine-learned model(s) 1. Online learning interface(s) 36 can facilitate reinforcement learning with human feedback (RLHF). Online learning interface(s) 36 can facilitate federated learning of machine-learned model(s) 1.

Model host 31 can execute machine-learned model(s) 1 to perform inference for various tasks using various types of data. For example, various different input(s) 2 and output(s) 3 can be used for various different tasks. In some implementations, input(s) 2 can be or otherwise represent image data. Machine-learned model(s) 1 can process the image data to generate an output. As an example, machine-learned model(s) 1 can process the image data to generate an image recognition output (e.g., a recognition of the image data, a latent embedding of the image data, an encoded representation of the image data, a hash of the image data, etc.). As another example, machine-learned model(s) 1 can process the image data to generate an image segmentation output. As another example, machine-learned model(s) 1 can process the image data to generate an image classification output. As another example, machine-learned model(s) 1 can process the image data to generate an image data modification output (e.g., an alteration of the image data, etc.). As another example, machine-learned model(s) 1 can process the image data to generate an encoded image data output (e.g., an encoded and/or compressed representation of the image data, etc.). As another example, machine-learned model(s) 1 can process the image data to generate an upscaled image data output. As another example, machine-learned model(s) 1 can process the image data to generate a prediction output.

In some implementations, the task is a computer vision task. In some cases, input(s) 2 includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some implementations, input(s) 2 can be or otherwise represent natural language data. Machine-learned model(s) 1 can process the natural language data to generate an output. As an example, machine-learned model(s) 1 can process the natural language data to generate a language encoding output. As another example, machine-learned model(s) 1 can process the natural language data to generate a latent text embedding output. As another example, machine-learned model(s) 1 can process the natural language data to generate a translation output. As another example, machine-learned model(s) 1 can process the natural language data to generate a classification output. As another example, machine-learned model(s) 1 can process the natural language data to generate a textual segmentation output. As another example, machine-learned model(s) 1 can process the natural language data to generate a semantic intent output. As another example, machine-learned model(s) 1 can process the natural language data to generate an upscaled text or natural language output (e.g., text or natural language data that is higher quality than the input text or natural language, etc.). As another example, machine-learned model(s) 1 can process the natural language data to generate a prediction output (e.g., one or more predicted next portions of natural language content).

In some implementations, input(s) 2 can be or otherwise represent speech data (e.g., data describing spoken natural language, such as audio data, textual data, etc.). Machine-learned model(s) 1 can process the speech data to generate an output. As an example, machine-learned model(s) 1 can process the speech data to generate a speech recognition output. As another example, machine-learned model(s) 1 can process the speech data to generate a speech translation output. As another example, machine-learned model(s) 1 can process the speech data to generate a latent embedding output. As another example, machine-learned model(s) 1 can process the speech data to generate an encoded speech output (e.g., an encoded and/or compressed representation of the speech data, etc.). As another example, machine-learned model(s) 1 can process the speech data to generate an upscaled speech output (e.g., speech data that is higher quality than the input speech data, etc.). As another example, machine-learned model(s) 1 can process the speech data to generate a textual representation output (e.g., a textual representation of the input speech data, etc.). As another example, machine-learned model(s) 1 can process the speech data to generate a prediction output.

In some implementations, input(s) 2 can be or otherwise represent latent encoding data (e.g., a latent space representation of an input, etc.). Machine-learned model(s) 1 can process the latent encoding data to generate an output. As an example, machine-learned model(s) 1 can process the latent encoding data to generate a recognition output. As another example, machine-learned model(s) 1 can process the latent encoding data to generate a reconstruction output. As another example, machine-learned model(s) 1 can process the latent encoding data to generate a search output. As another example, machine-learned model(s) 1 can process the latent encoding data to generate a reclustering output. As another example, machine-learned model(s) 1 can process the latent encoding data to generate a prediction output.

In some implementations, input(s) 2 can be or otherwise represent statistical data. Statistical data can be, represent, or otherwise include data computed and/or calculated from some other data source. Machine-learned model(s) 1 can process the statistical data to generate an output. As an example, machine-learned model(s) 1 can process the statistical data to generate a recognition output. As another example, machine-learned model(s) 1 can process the statistical data to generate a prediction output. As another example, machine-learned model(s) 1 can process the statistical data to generate a classification output. As another example, machine-learned model(s) 1 can process the statistical data to generate a segmentation output. As another example, machine-learned model(s) 1 can process the statistical data to generate a visualization output. As another example, machine-learned model(s) 1 can process the statistical data to generate a diagnostic output.

In some implementations, input(s) 2 can be or otherwise represent sensor data. Machine-learned model(s) 1 can process the sensor data to generate an output. As an example, machine-learned model(s) 1 can process the sensor data to generate a recognition output. As another example, machine-learned model(s) 1 can process the sensor data to generate a prediction output. As another example, machine-learned model(s) 1 can process the sensor data to generate a classification output. As another example, machine-learned model(s) 1 can process the sensor data to generate a segmentation output. As another example, machine-learned model(s) 1 can process the sensor data to generate a visualization output. As another example, machine-learned model(s) 1 can process the sensor data to generate a diagnostic output. As another example, machine-learned model(s) 1 can process the sensor data to generate a detection output.

In some implementations, machine-learned model(s) 1 can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g. one or more images or videos), the output comprises compressed visual data, and the task is a visual data compression task. In another example, the task may comprise generating an embedding for input data (e.g. input audio or visual data). In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

In some implementations, the task is a generative task, and machine-learned model(s) 1 can be configured to output content generated in view of input(s) 2. For instance, input(s) 2 can be or otherwise represent data of one or more modalities that encodes context for generating additional content.

In some implementations, the task can be a text completion task. Machine-learned model(s) 1 can be configured to process input(s) 2 that represent textual data and to generate output(s) 3 that represent additional textual data that completes a textual sequence that includes input(s) 2. For instance, machine-learned model(s) 1 can be configured to generate output(s) 3 to complete a sentence, paragraph, or portion of text that follows from a portion of text represented by input(s) 2.

In some implementations, the task can be an instruction following task. Machine-learned model(s) 1 can be configured to process input(s) 2 that represent instructions to perform a function and to generate output(s) 3 that advance a goal of satisfying the instruction function (e.g., at least a step of a multi-step procedure to perform the function). Output(s) 3 can represent data of the same or of a different modality as input(s) 2. For instance, input(s) 2 can represent textual data (e.g., natural language instructions for a task to be performed) and machine-learned model(s) 1 can process input(s) 2 to generate output(s) 3 that represent textual data responsive to the instructions (e.g., natural language responses, programming language responses, machine language responses, etc.). Input(s) 2 can represent image data (e.g., image-based instructions for a task to be performed, optionally accompanied by textual instructions) and machine-learned model(s) 1 can process input(s) 2 to generate output(s) 3 that represent textual data responsive to the instructions (e.g., natural language responses, programming language responses, machine language responses, etc.). One or more output(s) 3 can be iteratively or recursively generated to sequentially process and accomplish steps toward accomplishing the requested functionality. For instance, an initial output can be executed by an external system or be processed by machine-learned model(s) 1 to complete an initial step of performing a function. Multiple steps can be performed, with a final output being obtained that is responsive to the initial instructions.

In some implementations, the task can be a question answering task. Machine-learned model(s) 1 can be configured to process input(s) 2 that represent a question to answer and to generate output(s) 3 that advance a goal of returning an answer to the question (e.g., at least a step of a multi-step procedure to perform the function). Output(s) 3 can represent data of the same or of a different modality as input(s) 2. For instance, input(s) 2 can represent textual data (e.g., natural language instructions for a task to be performed) and machine-learned model(s) 1 can process input(s) 2 to generate output(s) 3 that represent textual data responsive to the question (e.g., natural language responses, programming language responses, machine language responses, etc.). Input(s) 2 can represent image data (e.g., image-based instructions for a task to be performed, optionally accompanied by textual instructions) and machine-learned model(s) 1 can process input(s) 2 to generate output(s) 3 that represent textual data responsive to the question (e.g., natural language responses, programming language responses, machine language responses, etc.). One or more output(s) 3 can be iteratively or recursively generated to sequentially process and accomplish steps toward answering the question. For instance, an initial output can be executed by an external system or be processed by machine-learned model(s) 1 to complete an initial step of obtaining an answer to the question (e.g., querying a database, performing a computation, executing a script, etc.). Multiple steps can be performed, with a final output being obtained that is responsive to the question.

In some implementations, the task can be an image generation task. Machine-learned model(s) 1 can be configured to process input(s) 2 that represent context regarding a desired portion of image content. The context can include text data, image data, audio data, etc. Machine-learned model(s) 1 can be configured to generate output(s) 3 that represent image data that depicts imagery related to the context. For instance, machine-learned model(s) 1 can be configured to generate pixel data of an image. Values for channel(s) associated with the pixels in the pixel data can be selected based on the context (e.g., based on a probability determined based on the context).

In some implementations, the task can be an audio generation task. Machine-learned model(s) 1 can be configured to process input(s) 2 that represent context regarding a desired portion of audio content. The context can include text data, image data, audio data, etc. Machine-learned model(s) 1 can be configured to generate output(s) 3 that represent audio data related to the context. For instance, machine-learned model(s) 1 can be configured to generate waveform data in the form of an image (e.g., a spectrogram). Values for channel(s) associated with pixels of the image can be selected based on the context. Machine-learned model(s) 1 can be configured to generate waveform data in the form of a sequence of discrete samples of a continuous waveform. Values of the sequence can be selected based on the context (e.g., based on a probability determined based on the context).

In some implementations, the task can be a data generation task. Machine-learned model(s) 1 can be configured to process input(s) 2 that represent context regarding a desired portion of data (e.g., data from various data domains, such as sensor data, image data, multimodal data, statistical data, etc.). The desired data can be, for instance, synthetic data for training other machine-learned models. The context can include arbitrary data type(s). Machine-learned model(s) 1 can be configured to generate output(s) 3 that represent data that aligns with the desired data. For instance, machine-learned model(s) 1 can be configured to generate data values for populating a dataset. Values for the data object(s) can be selected based on the context (e.g., based on a probability determined based on the context).

### Example Computing Systems and Devices

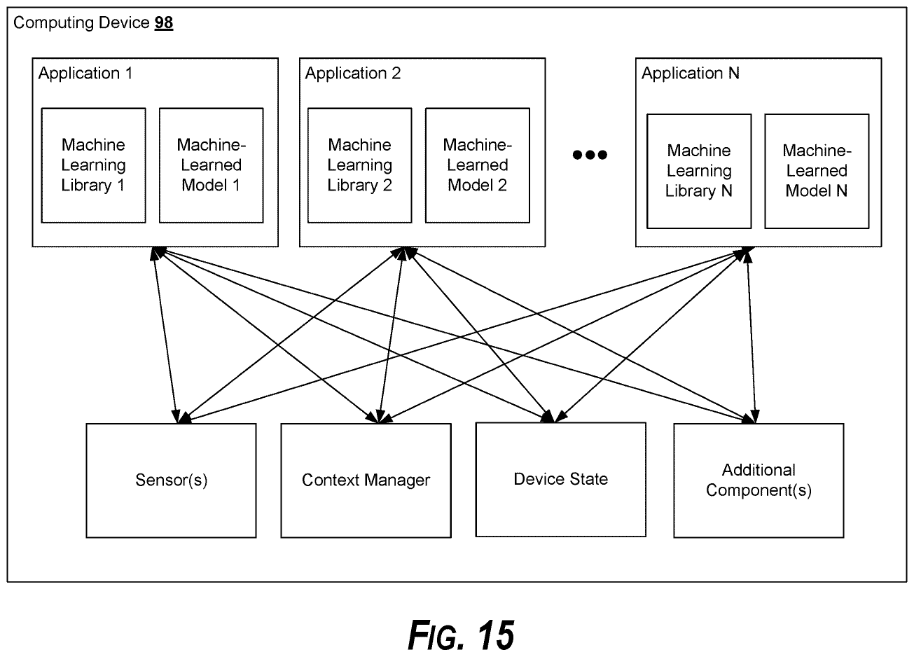

FIG. 14 is a block diagram of an example networked computing system that can perform aspects of example implementations of the present disclosure. The system can include a number of computing devices and systems that are communicatively coupled over a network 49. An example computing device 50 is described to provide an example of a computing device that can perform any aspect of the present disclosure (e.g., implementing model host 31, client(s) 32, or both). An example server computing system 60 is described as an example of a server computing system that can perform any aspect of the present disclosure (e.g., implementing model host 31, client(s) 32, or both). Computing device 50 and server computing system(s) 60 can cooperatively interact (e.g., over network 49) to perform any aspect of the present disclosure (e.g., implementing model host 31, client(s) 32, or both). Model development platform system 70 is an example system that can host or serve model development platform(s) 12 for development of machine-learned models. Third-party system(s) 80 are example system(s) with which any of computing device 50, server computing system(s) 60, or model development platform system(s) 70 can interact in the performance of various aspects of the present disclosure (e.g., engaging third-party tools, accessing third-party databases or other resources, etc.).

Network 49 can be any type of communications network, such as a local area network (e.g., intranet), wide area network (e.g., Internet), or some combination thereof and can include any number of wired or wireless links. In general, communication over network 49 can be carried via any type of wired or wireless connection, using a wide variety of communication protocols (e.g., TCP/IP, HTTP, SMTP, FTP), encodings or formats (e.g., HTML, XML), or protection schemes (e.g., VPN, secure HTTP, SSL). Network 49 can also be implemented via a system bus. For instance, one or more devices or systems of FIG. 14 can be co-located with, contained by, or otherwise integrated into one or more other devices or systems.

Computing device 50 can be any type of computing device, such as, for example, a personal computing device (e.g., laptop or desktop), a mobile computing device (e.g., smartphone or tablet), a gaming console or controller, a wearable computing device, an embedded computing device, a server computing device, a virtual machine operating on a host device, or any other type of computing device. Computing device 50 can be a client computing device. Computing device 50 can be an end-user computing device. Computing device 50 can be a computing device of a service provided that provides a service to an end user (who may use another computing device to interact with computing device 50).

Computing device 50 can include one or more processors 51 and a memory 52. Processor(s) 51 can be any suitable processing device (e.g., a processor core, a microprocessor, an ASIC, an FPGA, a controller, a microcontroller, etc.) and can be one processor or a plurality of processors that are operatively connected. Memory 52 can include one or more non-transitory computer-readable storage media, such as HBM, RAM, ROM, EEPROM, EPROM, flash memory devices, magnetic disks, etc., and combinations thereof. Memory 52 can store data 53 and instructions 54 which can be executed by processor(s) 51 to cause computing device 50 to perform operations. The operations can implement any one or multiple features described herein. The operations can implement example methods and techniques described herein.

Computing device 50 can also include one or more input components that receive user input. For example, a user input component can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, camera, LIDAR, a physical keyboard or other buttons, or other means by which a user can provide user input.

Computing device 50 can store or include one or more machine-learned models 55. Machine-learned models 55 can include one or more machine-learned model(s) 1, such as a sequence processing model 4. Machine-learned models 55 can include one or multiple model instance(s) 31-1. Machine-learned model(s) 55 can be received from server computing system(s) 60, model development platform system 70, third party system(s) 80 (e.g., an application distribution platform), or developed locally on computing device 50. Machine-learned model(s) 55 can be loaded into memory 52 and used or otherwise implemented by processor(s) 51. Computing device 50 can implement multiple parallel instances of machine-learned model(s) 55.

Server computing system(s) 60 can include one or more processors 61 and a memory 62. Processor(s) 61 can be any suitable processing device (e.g., a processor core, a microprocessor, an ASIC, an FPGA, a controller, a microcontroller, etc.) and can be one processor or a plurality of processors that are operatively connected. Memory 62 can include one or more non-transitory computer-readable storage media, such as HBM, RAM, ROM, EEPROM, EPROM, flash memory devices, magnetic disks, etc., and combinations thereof. Memory 62 can store data 63 and instructions 64 which can be executed by processor(s) 61 to cause server computing system(s) 60 to perform operations. The operations can implement any one or multiple features described herein. The operations can implement example methods and techniques described herein.

In some implementations, server computing system 60 includes or is otherwise implemented by one or multiple server computing devices. In instances in which server computing system 60 includes multiple server computing devices, such server computing devices can operate according to sequential computing architectures, parallel computing architectures, or some combination thereof.

Server computing system 60 can store or otherwise include one or more machine-learned models 65. Machine-learned model(s) 65 can be the same as or different from machine-learned model(s) 55. Machine-learned models 65 can include one or more machine-learned model(s) 1, such as a sequence processing model 4. Machine-learned models 65 can include one or multiple model instance(s) 31-1. Machine-learned model(s) 65 can be received from computing device 50, model development platform system 70, third party system(s) 80, or developed locally on server computing system(s) 60. Machine-learned model(s) 65 can be loaded into memory 62 and used or otherwise implemented by processor(s) 61. Server computing system(s) 60 can implement multiple parallel instances of machine-learned model(s) 65.

In an example configuration, machine-learned models 65 can be included in or otherwise stored and implemented by server computing system 60 to establish a client-server relationship with computing device 50 for serving model inferences. For instance, server computing system(s) 60 can implement model host 31 on behalf of client(s) 32 on computing device 50. For instance, machine-learned models 65 can be implemented by server computing system 60 as a portion of a web service (e.g., remote machine-learned model hosting service, such as an online interface for performing machine-learned model operations over a network on server computing system(s) 60). For instance, server computing system(s) 60 can communicate with computing device 50 over a local intranet or internet connection. For instance, computing device 50 can be a workstation or endpoint in communication with server computing system(s) 60, with implementation of machine-learned models 65 being managed by server computing system(s) 60 to remotely perform inference (e.g., for runtime or training operations), with output(s) returned (e.g., cast, streamed, etc.) to computing device 50. Machine-learned models 65 can work cooperatively or interoperatively with machine-learned models 55 on computing device 50 to perform various tasks.

Model development platform system(s) 70 can include one or more processors 71 and a memory 72. Processor(s) 71 can be any suitable processing device (e.g., a processor core, a microprocessor, an ASIC, an FPGA, a controller, a microcontroller, etc.) and can be one processor or a plurality of processors that are operatively connected. Memory 72 can include one or more non-transitory computer-readable storage media, such as HBM, RAM, ROM, EEPROM, EPROM, flash memory devices, magnetic disks, etc., and combinations thereof. Memory 72 can store data 73 and instructions 74 which can be executed by processor(s) 71 to cause model development platform system(s) 70 to perform operations. The operations can implement any one or multiple features described herein. The operations can implement example methods and techniques described herein. Example operations include the functionality described herein with respect to model development platform 12. This and other functionality can be implemented by developer tool(s) 75.

Third-party system(s) 80 can include one or more processors 81 and a memory 82. Processor(s) 81 can be any suitable processing device (e.g., a processor core, a microprocessor, an ASIC, an FPGA, a controller, a microcontroller, etc.) and can be one processor or a plurality of processors that are operatively connected. Memory 82 can include one or more non-transitory computer-readable storage media, such as HBM, RAM, ROM, EEPROM, EPROM, flash memory devices, magnetic disks, etc., and combinations thereof. Memory 82 can store data 83 and instructions 84 which can be executed by processor(s) 81 to cause third-party system(s) 80 to perform operations. The operations can implement any one or multiple features described herein. The operations can implement example methods and techniques described herein. Example operations include the functionality described herein with respect to tools and other external resources called when training or performing inference with machine-learned model(s) 1, 4, 16, 20, 55, 65, etc. (e.g., third-party resource(s) 85).

FIG. 14 illustrates one example arrangement of computing systems that can be used to implement the present disclosure. Other computing system configurations can be used as well. For example, in some implementations, one or both of computing system 50 or server computing system(s) 60 can implement all or a portion of the operations of model development platform system 70. For example, computing system 50 or server computing system(s) 60 can implement developer tool(s) 75 (or extensions thereof) to develop, update/train, or refine machine-learned models 1, 4, 16, 20, 55, 65, etc. using one or more techniques described herein with respect to model alignment toolkit 17. In this manner, for instance, computing system 50 or server computing system(s) 60 can develop, update/train, or refine machine-learned models based on local datasets (e.g., for model personalization/customization, as permitted by user data preference selections).

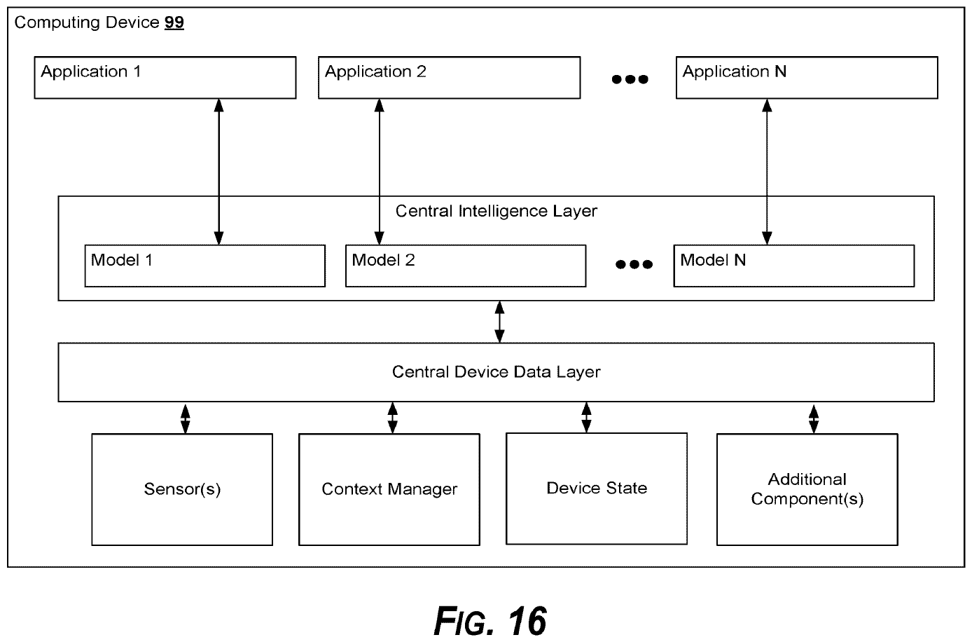

FIG. 15 is a block diagram of an example computing device 98 that performs according to example embodiments of the present disclosure. Computing device 98 can be a user computing device or a server computing device (e.g., computing device 50, server computing system(s) 60, etc.). Computing device 98 can implement model host 31. For instance, computing device 98 can include a number of applications (e.g., applications 1 through N). Each application can contain its own machine learning library and machine-learned model(s). For example, each application can include a machine-learned model. Example applications include a text messaging application, an email application, a dictation application, a virtual keyboard application, a browser application, etc. As illustrated in FIG. 15, each application can communicate with a number of other components of the computing device, such as, for example, one or more sensors, a context manager, a device state component, or additional components. In some implementations, each application can communicate with each device component using an API (e.g., a public API). In some implementations, the API used by each application is specific to that application.

FIG. 16 is a block diagram of an example computing device 99 that performs according to example embodiments of the present disclosure. Computing device 99 can be the same as or different from computing device 98. Computing device 99 can be a user computing device or a server computing device (e.g., computing device 50, server computing system(s) 60, etc.). Computing device 98 can implement model host 31. For instance, computing device 99 can include a number of applications (e.g., applications 1 through N).

Each application can be in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer can include a number of machine-learned models. For example, as illustrated in FIG. 16, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of computing device 99.

The central intelligence layer can communicate with a central device data layer. The central device data layer can be a centralized repository of data for computing device 99. As illustrated in FIG. 16, the central device data layer can communicate with a number of other components of the computing device, such as, for example, one or more sensors, a context manager, a device state component, or additional components. In some implementations, the central device data layer can communicate with each device component using an API (e.g., a private API).

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

Aspects of the disclosure have been described in terms of illustrative embodiments thereof. Any and all features in the following claims can be combined or rearranged in any way possible, including combinations of claims not explicitly enumerated in combination together, as the example claim dependencies listed herein should not be read as limiting the scope of possible combinations of features disclosed herein. Accordingly, the scope of the present disclosure is by way of example rather than by way of limitation, and the subject disclosure does not preclude inclusion of such modifications, variations or additions to the present subject matter as would be readily apparent to one of ordinary skill in the art. Moreover, terms are described herein using lists of example elements joined by conjunctions such as “and,” “or,” “but,” etc. It should be understood that such conjunctions are provided for explanatory purposes only. Clauses and other sequences of items joined by a particular conjunction such as “or,” for example, can refer to “and/or,” “at least one of”, “any combination of” example elements listed therein, etc. Terms such as “based on” should be understood as “based at least in part on.”

The term “can” should be understood as referring to a possibility of a feature in various implementations and not as prescribing an ability that is necessarily present in every implementation. For example, the phrase “X can perform Y” should be understood as indicating that, in various implementations, X has the potential to be configured to perform Y, and not as indicating that in every instance X must always be able to perform Y. It should be understood that, in various implementations, X might be unable to perform Y and remain within the scope of the present disclosure.

The term “may” should be understood as referring to a possibility of a feature in various implementations and not as prescribing an ability that is necessarily present in every implementation. For example, the phrase “X may perform Y” should be understood as indicating that, in various implementations, X has the potential to be configured to perform Y, and not as indicating that in every instance X must always be able to perform Y. It should be understood that, in various implementations, X might be unable to perform Y and remain within the scope of the present disclosure.

1. A computer-implemented method for generating health-related predictions based on individual-level mobile health data, the method comprising:
obtaining, by a computing system comprising one or more computing devices, mobile health data associated with an individual, wherein the mobile health data comprises sensor data collected by a user computing device of the individual or derived from sensor data collected by the user computing device of the individual;
processing, by the computing system, the mobile health data with a sequence processing model to generate, as an output of the sequence processing model, a health-related prediction for the individual; and
providing, by the computing system, the health-related prediction as an output.

2. The computer-implemented method of claim 1, wherein the user computing device is configured to be worn.

3. The computer-implemented method of claim 1, wherein the user computing device comprises a wrist-worn watch.

4. The computer-implemented method of claim 1, wherein processing, by the computing system, the mobile health data with the sequence processing model comprises:
appending, by the computing system, the mobile health data to a prompt to form a combined prompt, wherein the prompt comprises a textual instruction to predict the health-related prediction; and
processing, by the computing system, the combined prompt with the sequence processing model to generate, as the output of the sequence processing model, the health-related prediction for the individual.

5. The computer-implemented method of claim 4, wherein the prompt further comprises additional feature data descriptive of the individual.

6. The computer-implemented method of claim 1, wherein at least some of the mobile health data comprises time series sensor data collected by one or more sensors of the user computing device.

7. The computer-implemented method of claim 6, wherein at least some of the time series sensor data was passively collected by the user computing device.

8. The computer-implemented method of claim 1, wherein at least some of the mobile health data comprises textual data.

9. The computer-implemented method of claim 1, wherein at least some of the mobile health data comprises sensor data and wherein, prior to processing the mobile health data with the sequence processing model, the method further comprises transforming, by the computing system, the sensor data into embeddings using a time-series encoder model.

10. The computer-implemented method of claim 1, wherein the mobile health data comprises one or more of the following items derived from sensor data collected by the user computing device: sleep score; light sleep time duration; REM sleep time duration; deep sleep time duration; total sleep time duration; fall asleep time; time awake time duration; sleep efficiency; percent of sleep goal; wakeup count;
heart rate data; nap duration time duration; number of naps; resting heart rate data; root mean square of successive differences between normal heartbeats (RMSSD); respiratory rate; fat-burn zone time duration; cardio zone time duration; peak zone time duration; training impulse (TRIMP); and total steps taken.

11. The computer-implemented method of claim 1, wherein the mobile health data comprises one or more of the following: heart rate variability root mean square of successive differences; respiratory rate; or resting heart rate.

12. The computer-implemented method of claim 1, wherein the health-related prediction comprises a prediction of a user-submitted report.

13. The computer-implemented method of claim 1, wherein the health-related prediction comprises a health coaching message.

14. The computer-implemented method of claim 1, wherein the sequence processing model has been trained on a dataset comprising professional examination data.

15. The computer-implemented method of claim 1, wherein the sequence processing model has been trained on a dataset comprising patient-reported outcome data.

16. The computer-implemented method of claim 1, wherein the sequence processing model has been trained on a dataset comprising case studies, wherein at least one of the case studies comprises a machine-generated response that has been validated by a human expert.

17. A computing system comprising one or more processors and one or more non-transitory computer-readable media that collectively store processor-executable instructions for performing operations, the operations comprising:
receiving a query relating to a particular individual;
obtaining health features associated with the particular individual, wherein the health features comprise one or more features from each of a plurality of modalities, and wherein, for at least one of the plurality of modalities, the health features are sensor data collected by a user computing device of the individual or are derived from sensor data collected by the user computing device of the individual;
generating a respective representation of each of the multi-modal health features as a respective set of one or more tokens in a natural language embedding space;
generating an input sequence of tokens that comprises one or more tokens in the natural language embedding space representing the query and the respective sets of one or more tokens in the natural language embedding space that represent each of the one or more features from each of the plurality of modalities;
processing the input sequence of tokens using a sequence processing model to generate a model output; and
generating, from the model output, a health-related prediction that is responsive to the query.

18. The computing system of claim 17, wherein the health features are health tracker metrics collected by the user computing device.

19. A non-transitory computer-readable storage medium having stored thereon processor-executable instructions that, when executed by one or more processors of a computer system, cause the computer system to perform operations comprising:
obtaining a set of health-related data from one or more data sources;
prompting a large language model with the set of health-related data to generate an initial output comprising one or more health-related predictions;
receiving edits from one or more human experts to the initial output;
submitting the edited initial output to a clinical lead for review and validation, thereby creating a validated response;
combining the validated response with the set of health-related data to form a training tuple; and
training a sequence processing model on the training tuple, wherein the sequence processing model is configured to process and analyze multi-modal health data to generate personalized health-related predictions and responses when executed.

20. The non-transitory computer-readable storage medium of claim 19, wherein the set of health-related data comprises mobile health data that comprises sensor data collected by a user computing device of the individual or derived from sensor data collected by the user computing device of the individual.
